(19) United States
(12) Patent Application Publication
(10) Pub. No.: US 2023/0265211 A1
(43) Pub. Date: Aug. 24, 2023

(54) ANTI-PROTEIN S SINGLE-DOMAIN ANTIBODIES AND POLYPEPTIDES COMPRISING THEREOF

(71) Applicants: INSERM (INSTITUT NATIONAL DE LA SANTÉ ET DE LS RECHERCHE MÉDICALE), Paris (FR); ASSISTANCE PUBLIQUE-HÔPITAUX DE PARIS (APHP), Paris (FR); UNIVERSITÉ PARIS-SACLAY, Gif-Sur-Yvette (FR)

(72) Inventors: François SALLER, Le Kremlin Bicetre Cedex (FR); Cécile DENIS, Le Kremlin Bicetre (FR); Delphine BORGEL, Le Kremlin Bicetre Cedex (FR); Frédéric BORGEL, Le Kremlin Bicetre Cedex (FR); Oliver CHRISTOPHE, Le Kremlin-Bicetre (FR); Peter LENTING, Le Kremlin-Bicetre (FR)

(21) Appl. No.: 18/012,322

(22) PCT Filed: Jun. 28, 2021
(86) PCT No.: PCT/EP2021/067746

§ 371 Date: Dec. 22, 2022

(30) Foreign Application Priority Data
Jun. 29, 2020 (EP) 20305723.7

(51) Int. Cl. C07K 16/36; A61P 7/02

(52) U.S. Cl. CPC C07K 16/36; A61P 7/02; A61K 2039/505

(57) ABSTRACT

Vitamin K-dependent Protein S (PS) is a natural anticoagulant acting as a non-enzymatic cofactor for both activated protein C (APC) and tissue factor pathway inhibitor (TFPI). The inventors identify an anti-PS nanobody that very surprisingly enhances the APC-cofactor activity of PS through unknown mechanisms. Very interestingly, this nanobody exerts an antithrombotic effect in injured mesenteric microvessels of mice. As a consequence, it10 constitutes a novel class of antithrombotic agents that could be used for the treatment of acute microthrombosis in pathological states such as sepsis, COVID-19, distal microvascular thrombosis induced by stroke, or sickle-cell disease. Thus, the present invention relates to isolated single-domain antibodies (sdAb) directed against protein S (PS) and polypeptides comprising thereof.

### FIELD OF THE INVENTION

The present invention relates to anti-protein S (PS) conformational single-domain antibodies and polypeptides comprising thereof and their uses thereof in particular in the therapeutic field.

### BACKGROUND OF THE INVENTION

Vitamin K-dependent Protein S (PS) is a natural anticoagulant acting as a non-enzymatic cofactor for both activated protein C (APC) and tissue factor pathway inhibitor (TFPI). Indeed, PS has been historically described as being able to enhance the proteolytic activity of APC towards activated factor V (FVa) and activated factor VIII (FVIIIa), which very effectively limits thrombin generation. PS has also largely been reported to enhance the inhibitory effects of TFPI-α on FXa (Hackeng et al. 2006), although the physiological relevance of such a cofactor activity is not precisely known. In addition, PS has more recently been described as a direct inhibitor of activated factor IX (FIXa) (Plautz et al. 2018). The physiological importance of PS is attested by the clinical manifestations observed in PS-deficient patients. Whereas mild deficiency in PS is associated with an increased risk of venous thrombosis, severe PS deficiency results in a dramatic and life-threatening prothrombotic phenotype (i.e. purpura fulminans with microvascular thromboses especially in dermal vessels and disseminated intravascular coagulation). In mice, total deficiency in PS is embryonically lethal due to severe thrombotic coagulopathy and massive intracerebral hemorrhages (Saller et al. 2009; Burstyn-Cohen et al. 2009). In hemophilic mice, PS is highly expressed in the joint, which might partly explain the highly anticoagulant environment observed in the hemophilic joint (Prince, Bologna et al. 2018). Interestingly, absence of PS or its pharmacological inhibition with polyclonal antibodies result in an important reduction of hemarthroses.

In this context, inventors aimed at developing nanobodies directed against PS as original and potent tools for modulating the anticoagulant activity of PS. Nanobodies, or single-domain antibodies (sdAb), are the variable region (VHH) of heavy-chain-only antibodies (HcAb) that are found in camelids. Despite their small size (15 kDa), the isolated nanobodies can fully recognize their cognate antigens. In addition, due to their small size and physicochemical properties they have a variety of advantages over conventional immunoglobulins. For example, they retain a high stability even though they are isolated from the remainder of their HcAb of origin. Also, they can be soluble at high concentrations, and they are considered to exhibit an excellent tissue penetration in vivo. Consequently, nanobodies have emerged as a novel and promising class of therapeutic antibodies. Furthermore, they can be expressed in E. coli and they can readily be combined with other nanobodies to generate multivalent or multispecific species. One of their advantages is that, as compared to classical monocolonal antibodies, nanobodies can recognize cryptic epitopes through their protruding complementary determining region 3 (CDR3). Inventors thus reasoned that anti-PS nanobodies might allow to identify original antibodies that could modulate the anticoagulant activity of PS in an unanticipated manner.

To date, there is still a need for developing safer antithrombotic agents. Indeed, currently used antithrombotic drugs such as antiplatelet (e.g. aspirin and clopidogrel) or anticoagulant (e.g. heparin derivatives, warfarin) drugs are associated with an increased risk of bleedings, as they interfere with the physiological hemostatic response. In contrast, it is possible that enhancing the anticoagulant activity of APC with anti-PS nanobody might have a minor impact on hemostasis and thus might not be associated with an increased risk of bleeding.

Physiological agents such as high-density lipoproteins (Griffin et al. 1999; Fernandez et al. 2015), cardiolipin (Fernandez et al. 2000) or skeletal muscle myosin (Heeb et al. 2019) have been reported to enhance the anticoagulant activity of APC. However, no pharmaceutical agent has yet been developed to enhance the anticoagulant activity of APC. Interestingly, a pharmaceutical activator of protein C is currently studied as a potential antithrombotic agent. This activator is a thrombin variant W215A/E217A (WE thrombin or AB002) which loses its procoagulant properties but is still able to activate protein C to APC (Cantwell et al. 2000). Whereas systemic administration of exogenous APC or systemic activation of protein C by high levels of soluble thrombomodulin in a patient (Dargaud et al. Blood 2015) are associated with an increased bleeding tendency, the administration of AB002 only modestly impairs hemostasis (Gruber et al. 2002). This is likely due, at least in part, to a localized action of AB002 on the thrombus surface where APC might be generated in situ by AB002, with limited escape of APC into the circulation (Gruber et al. 2007). This would explain why AB002 has been described to potently interrupt thrombus propagation in various animal models of thrombosis (Gruber et al. 2002; Tucker et al. Blood 2020), without profound systemic anticoagulant effect.

The nanobodies directed against PS developed by the inventors could be proposed in the treatment of acute pathologies, such as sepsis or stroke, in which microvascular thromboses play a major pathogenic role.

### SUMMARY OF THE INVENTION

To identify anti-PS nanobodies, inventors took advantage of a platform developed at the UMR_S1176 allowing us to screen a large library of nanobodies generated from a llama immunized with PS. They identify an anti-PS nanobody that very surprisingly enhances the APC-cofactor activity of PS through unknown mechanisms. Very interestingly, this nanobody exerts an antithrombotic effect in injured mesenteric microvessels of mice. As a consequence, it constitutes a novel class of antithrombotic agents that could be used for the treatment of acute microthrombosis in pathological states such as sepsis, COVID-19, or distal microvascular thrombosis induced by stroke.

Thus, the present invention refers to an isolated single-domain antibodies (sdAb) directed against protein S (PS) and polypeptides comprising thereof. In particular, the present invention is defined by the claims.

### DETAILED DESCRIPTION OF THE INVENTION

Protein S (PS) is a natural anticoagulant acting as a cofactor for activated protein C (APC) and tissue-factor pathway inhibitor (TFPI). Inventors hypothesized that modulating PS activities would be an efficient approach in the treatment of coagulation disorders. To this aim, inventors generated an immune library of single-domain antibodies (sdAb) from a llama immunized with recombinant human PS (rhPS), and selected sdAb targeting PS by phage display.

Inventors identified sdAb strongly binding PS and exhibiting anticoagulant effect in vitro and antithrombotic effect in vivo.

### Definition

As used herein, the term “Protein S” or “PS” has its general meaning in the art and refers to a vitamin K-dependent plasma glycoprotein mainly synthesized in the liver. In the circulation, Protein S exists in two forms: a free form and a complex form bound to complement protein C4b-binding protein (C4BP). In humans, protein S is encoded by the PSI gene. Protein S is a natural anticoagulant acting as a non-enzymatic cofactor for both activated protein C (APC) and tissue factor pathway inhibitor (TFPI). Indeed, PS has been historically described as being able to enhance the proteolytic activity of APC towards activated factor V (FVa) and activated factor VIII (FVIIIa), which very effectively limits thrombin generation. PS has also largely been reported to enhance the inhibitory effects of TFPI-α on FXa (Hackeng et al. 2006), although the physiological relevance of such a cofactor activity is not precisely known. In addition, PS has more recently been described as a direct inhibitor of activated factor IX (FIXa) (Plautz et al. 2018).

As used herein the term “single-domain antibody” has its general meaning in the art and refers to the single heavy chain variable domain of antibodies of the type that can be found in Camelid mammals which are naturally devoid of light chains. Such single-domain antibodies are also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. The nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins. Camelid nanobodies are useful as reagents to detect antigens that are otherwise cryptic using classical immunological techniques, and as possible therapeutic agents. Thus yet another consequence of small size is that a nanobody can exert its biological effects as a result of binding to a specific site in a groove or narrow cleft of a target protein, and hence can serve in a capacity that more closely resembles the function of a classical low molecular weight drug than that of a classical antibody. The low molecular weight and compact size further result in nanobodies being extremely thermostable, stable to extreme pH and to proteolytic digestion, and poorly antigenic. Another consequence is that nanobodies readily move from the circulatory system into tissues, and some of them can even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. The amino-acid sequence and structure of a single-domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 2” or “CDR2” and as “Complementarity Determining Region 3” or “CDR3”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the invention, the amino-acid residues of the single-domain antibody are numbered according to the general numbering for VH domains given by the International ImMunoGeneTics information system aminoacid numbering (http://imgt.cines.fr/).

As used herein, the term “amino-acid sequence” has its general meaning and is a sequence of amino acids that confers to a protein its primary structure. According to the invention, the amino-acid sequence may be modified with one, two or three conservative amino acid substitutions, without appreciable loss of interactive binding capacity. By “conservative amino acid substitution”, it is meant that an amino acid can be replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., glycine, cysteine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

According to the invention a first amino-acid sequence having at least 70% of identity with a second amino-acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; or 99% of identity with the second amino acid sequence. Amino-Sacid sequence identity is typically determined using a suitable sequence alignment algorithm and default parameters, such as BLAST P (Karlin and Altschul, 1990).

According to the meaning of the present invention, the “identity” is calculated by comparing two aligned sequences in a comparison window. The sequence alignment allows determining the number of positions (nucleotides or amino acids) in common for the two sequences in the comparison window. The number of positions in common is therefore divided by the total number of positions in the comparison window and multiplied by 100 to obtain the identity percentage. The determination of the identity percentage of sequence can be made manually or thanks to well-known computer programs.

As used herein, the terms “purified” and “isolated” relate to the sdAb of the invention and mean that the sdAb is present in the substantial absence of other biologic macromolecules of the same type. The term “purified” as used here means preferably that at least 75% in weight, more preferably at least 85% in weight, even more preferably at least 95% in weight, and the more preferably at least 98% in weight of antibody, compared to the total weight of macromolecules present.

As used herein, the term “nucleic acid molecule” has its general meaning in the art and refers to a DNA or RNA molecule

As used herein, the term “specifically binds to” means that an antibody only binds to the antigen of interest, e.g. Protein S (PS), as assessed using either recombinant forms of the proteins, epitopes therein, or native proteins present on the surface of isolated target cells and does not exhibit cross-reactivity to other antigens.

### Single Domain Antibodies and Polypeptide

The sequences of interest in the present application are indicated in the following Table 1:

| | SEQ | |
|---|---|---|
| | ID | |
| Name | NO: | SEQUENCE |
| PS003 CDR1 | 1 | SGRTFSSYA |
| PS003 CDR2 | 2 | ISYNGGRT |
| PS003 CDR3 | 3 | AANPRMWGSVDFRSW |
| PS003 | 4 | QVQLQESGGGLVQAGGSLRLSCAASGRTFSS |
| | | YAMGWVRQAPGKEREFVAAISYNGGRTNYAD |
| | | SVKGRFTISRDNAKNTGYLQMNSLKPEDTAV |
| | | YYCAANPRMWGSVDFRSWGQGTQVTVSS |
| PS003biv | 5 | QVQLQESGGGLVQAGGSLRLSCAASGRTFSS |
| | | YAMGWVRQAPGKEREFVAAISYNGGRTNYAD |
| | | SVKGRFTISRDNAKNTGYLQMNSLKPEDTAV |
| | | YYCAANPRMWGSVDFRSWGQGTQVTVSSGGG |
| | | SGGGSGGGSGGGSQVQLQESGGGLVQAGGSL |
| | | RLSCAASGRTFSSYAMGWVRQAPGKEREFVA |
| | | AISYNGGRTNYADSVKGRFTISRDNAKNTGY |
| | | LQMNSLKPEDTAVYYCAANPRMWGSVDFRSW |
| | | GQGTQVTVSS |

In a first aspect, the invention relates to an isolated single-domain antibody (sdAb) directed against protein S (PS).

In a first aspect, the invention relates to an isolated single-domain antibody (sdAb) binding specifically to protein S (PS).

In some embodiments, the isolated single domain antibody according to the invention is a PS agonist antibody.

As used herein, a «PS agonist» antibody refers to an antibody that exhibit the activity of PS. According to the invention, a «PS agonist» antibody refers to an antibody being able to enhance the APC-cofactor activity of PS, i.e. to enhance the proteolytic activity of APC towards activated factor V (FVa) and activated factor VIII (FVIIIa). According to the invention, a «PS agonist» antibody refers to an antibody being able to enhance the anticoagulant activity of protein S

Thus, in some embodiments, the isolated single domain antibody according to the invention of the invention enhances the APC-cofactor activity of PS.

In some embodiments, the isolated single domain antibody according to the invention bound to either recombinant or plasma-derived human PS and did not significantly interfere with their TFPI-cofactor activity.

According to the invention, the single-domain antibody directed against PS enhances the APC-cofactor activity of PS.

In some embodiments, the isolated single domain antibody according to the invention exhibit antithrombotic activity.

As used herein, the term “antithrombotic activity” has its general meaning in the art and refers to activity that reduces the formation of blood clots. According to the invention, the isolated single domain antibody reduces the formation of blood clots and/or dissolve the blood clots.

Tests for determining the capacity of an antibody to exhibit antithrombotic activity are well known to the person skilled in the art. Tests for determining the capacity of an antibody to specifically enhance the APC-cofactor activity of PS are well known to the person skilled in the art and include clot-based assay such as Prothrombin time (PT) assay, activated partial thromboplastin time (APTT) assay (see FIG. 8C-8D), specific one-stage clotting assays, calibrated automated thrombinography (CAT) or other thrombin generation assays, FVa inactivation assays (see FIG. 9) and FVIIIa inactivation assays, and TFPIα-cofactor activity assays (see FIG. 10).

In particular, the invention relates to an isolated single-domain antibody (sdAb) comprising a CDR1 having a sequence set forth as SEQ ID NO: 1, a CDR2 having a sequence set forth as SEQ ID NO:2 and a CDR3 having a sequence set forth as SEQ ID NO:3. (“PS003 derivative”).

In some embodiments, the isolated single domain antibody according to the invention has at least 70% of identity with sequence set forth as SEQ ID NO:4 (“PS003 derivative”).

In some embodiments, the isolated single domain antibody according to the invention has at least 70% of identity with sequence set forth as SEQ ID NO:4 and comprises the sequences CDR1, CDR2, CDR3 set forth as SEQ ID NO:1, SEQ ID NO:2 and SEQ ID NO:3.

In some embodiments, the isolated single domain antibody according to the invention comprises the sequence set forth as SEQ ID NO:4 (“PS003”).

In some embodiments, the isolated single domain antibody according to the invention has the sequence set forth as SEQ ID NO:4.

It should be further noted that the sdAb “PS003” enhances the APC-cofactor activity of PS.

It should be further noted that the sdAb “PS003” bound to either recombinant or plasma-derived human PS and did not significantly interfere with their TFPI-cofactor activity.

In some embodiments, the isolated single-domain antibody is a “humanized” single domain antibody.

As used herein the term “humanized” refers to a single-domain antibody of the invention wherein an amino-acid sequence that corresponds to the amino-acid sequence of a naturally occurring VHH domain has been “humanized”, i.e. by replacing one or more amino-acid residues in the amino-acid sequence of said naturally occurring VHH sequence (and in particular in the framework sequences) by one or more of the amino-acid residues that occur at the corresponding position(s) in a VH domain from a conventional chain antibody from a human being. Methods for humanizing single-domain antibodies are well known in the art. Typically, the humanizing substitutions should be chosen such that the resulting humanized single-domain antibodies still retain the favourable properties of single-domain antibodies of the invention. The one skilled in the art is able to determine and select suitable humanizing substitutions or suitable combinations of humanizing substitutions.

In some embodiments, the single-domain of the invention is conjugated with further therapeutic agents used to treat hypercoagulable disorders.

A further aspect of the invention refers to a cross-competing single-domain antibody which cross-competes for binding PS with the single-domain antibody of the invention. In some embodiment, the cross-competing single-domain antibody of the present invention cross-competes for binding PS with the single-domain antibody comprising a CDR1 having a sequence set forth as SEQ ID NO: 1, a CDR2 having a sequence set forth as SEQ ID NO:2 and a CDR3 having a sequence set forth as SEQ ID NO:3.

In some embodiment, the cross-competing single-domain antibody of the present invention cross-competes for binding PS with the single-domain antibody comprising or consisting in the sequence set forth as SEQ ID NO:4.

As used herein, the term “cross-competes” refers to single-domain antibodies which share the ability to bind to a specific region of an antigen. In the present disclosure the single-domain antibody that “cross-competes” has the ability to interfere with the binding of another single-domain antibody for the antigen in a standard competitive binding assay. Such a single-domain antibody may, according to non-limiting theory, bind to the same or a related or nearby (e.g., a structurally similar or spatially proximal) epitope as the single-domain antibody with which it competes. Cross-competition is present if single-domain antibody A reduces binding of single-domain antibody B at least by 60%, specifically at least by 70% and more specifically at least by 80% and vice versa in comparison to the positive control which lacks one of said single-domain antibodies. As the skilled artisan appreciates competition may be assessed in different assay set-ups. One suitable assay involves the use of the Biacore technology (e.g., by using the BIAcore 3000 instrument (Biacore, Uppsala, Sweden)), which can measure the extent of interactions using surface plasmon resonance technology. Another assay for measuring cross-competition uses an ELISA-based approach. Furthermore, a high throughput process for “binning” antibodies based upon their cross-competition is described in International Patent Application No. WO2003/48731.

According to the present invention, the cross-competing antibody as above described retain the activity of the single antibody which comprises a CDR1 having a sequence set forth as SEQ ID NO:1, a CDR2 having a sequence set forth as SEQ ID NO:2 and a CDR3 having a sequence set forth as SEQ ID NO:3.

According to the present invention, the cross-competing antibody as above described retain the activity of the single-antibody comprising or consisting in the sequence set forth as SEQ ID NO:4.

Thus, in some embodiment, the cross-competing single-domain antibody of the present invention is a PS agonist antibody.

In some embodiment, the cross-competing single-domain antibody of the present invention enhances the APC-cofactor activity of PS.

In some embodiments, the he cross-competing single-domain antibody of the present invention bound to either recombinant or plasma-derived human PS and did not significantly interfere with their TFPI-cofactor activity.

A further aspect of the invention refers to a polypeptide comprising at least one single-domain antibody of the invention.

Typically, the polypeptide of the invention comprises a single-domain antibody of the invention, which is fused at its N terminal end, at its C terminal end, or both at its N terminal end and at its C terminal end to at least one further amino-acid sequence, i.e. so as to provide a fusion protein. According to the invention the polypeptides that comprise a sole single-domain antibody are referred to herein as “monovalent” polypeptides. Polypeptides that comprise or essentially consist of two or more single-domain antibodies according to the invention are referred to herein as “multivalent” polypeptides. Typically, multivalent polypeptides could be: biparatopic antibody, trivalent antibody or quadrivalent antibody.

In some embodiment, the polypeptide of the invention enhances the APC-cofactor activity of PS.

In some embodiments, the polypeptide of the invention bound to either recombinant or plasma-derived human PS and did not significantly interfere with their TFPI-cofactor activity.

In some embodiments, the polypeptide comprises at least one single-domain antibody of the invention and at least one other binding unit (i.e. directed against another epitope, antigen, target, protein or polypeptide), which is typically also a single-domain antibody. Such a polypeptide is referred to herein as “multispecific” polypeptide; in opposition to a polypeptide comprising the same single-domain antibodies (“monospecific” polypeptide). Thus, in some embodiments, the polypeptide of the invention may also provide at least one further binding site directed against any desired protein, polypeptide, antigen, antigenic determinant or epitope. Said binding site is directed against to the same protein, polypeptide, antigen, antigenic determinant or epitope for which the single-domain antibody of the invention is directed against, or may be directed against a different protein, polypeptide, antigen, antigenic determinant or epitope) from the single-domain antibody of the invention.

In some embodiments, the polypeptide of the present invention comprises at least one single-domain antibody comprising a CDR1 having a sequence set forth as SEQ ID NO: 1, a CDR2 having a sequence set forth as SEQ ID NO:2 and a CDR3 having a sequence set forth as SEQ ID NO:3.

In some embodiments, the polypeptide of the present invention comprises at least two single-domain antibodies comprising a CDR1 having a sequence set forth as SEQ ID NO: 1, a CDR2 having a sequence set forth as SEQ ID NO:2 and a CDR3 having a sequence set forth as SEQ ID NO:3.

In some embodiments, the polypeptide of the present invention comprises 2, 3, 4 or 5 single-domain antibodies comprising a CDR1 having a sequence set forth as SEQ ID NO: 1, a CDR2 having a sequence set forth as SEQ ID NO:2 and a CDR3 having a sequence set forth as SEQ ID NO:3.

In some embodiments, the polypeptide of the present invention comprises at least two single-domain antibodies having at least 70% of identity with sequence set forth as SEQ ID NO:4.

In some embodiments, the polypeptide of the present invention comprises at least two single-domain antibodies having at least 70% of identity with sequence set forth as SEQ ID NO:4 and comprising CDR1, CDR2, CDR3 set forth as SEQ ID NO:1, SEQ ID NO:2 and SEQ ID NO:3.

In some embodiments, the polypeptide of the present invention comprises at least two single-domain antibodies having a sequence set forth as SEQ ID NO:4.

In some embodiments, the polypeptide of the present invention comprises 2, 3, 4 or 5 single-domain antibodies having a sequence set forth as SEQ ID NO:4.

In some embodiments, the polypeptide of the present invention comprises a sequence having at least 70% of identity with sequence set forth as SEQ ID NO:5 (“PS003Biv derivative”)

In some embodiments, the polypeptide of the present invention comprises a sequence set forth as SEQ ID NO:5 (“PS003Biv”).

In some embodiments, the polypeptide of the present invention has a sequence set forth as SEQ ID NO:5 (“PS003Biv”).

In some embodiments, the single-domain antibodies of the polypeptide of the present invention can be linked to each other directly (i.e. without use of a linker) or via a linker. The linker is typically a linker peptide and will, according to the invention, be selected so as to allow binding of the two single-domain antibodies to each of their at least two different epitopes of PS. Suitable linkers inter alia depend on the epitopes and, specifically, the distance between the epitopes on PS to which the single-domain antibodies bind, and will be clear to the skilled person based on the disclosure herein, optionally after some limited degree of routine experimentation. Also, when the two single-domain antibodies that bind to PS may also be linked to each other via a third single-domain antibody (in which the two single-domain antibodies may be linked directly to the third-domain antibody or via suitable linkers). Such a third single-domain antibody may for example be a single-domain antibody that provides for an increased half-life. For example, the latter single-domain antibody may be a single-domain antibody that is capable of binding to a (human) serum protein such as (human) serum albumin or (human) transferrin, as further described herein. In some embodiments, two or more single-domain antibodies that bind to PS are linked in series (either directly or via a suitable linker) and the third (single) single-domain antibody (which may provide for increased half-life, as described above) is connected directly or via a linker to one of these two or more aforementioned single-domain antibodies. Suitable linkers are described herein in connection with specific polypeptides of the invention and may—for example and without limitation—comprise an amino-acid sequence, which amino-acid sequence preferably has a length of 9 or more amino acids, more preferably at least 17 amino acids, such as about 20 to 40 amino acids. However, the upper limit is not critical but is chosen for reasons of convenience regarding e.g. biopharmaceutical production of such polypeptides. The linker sequence may be a naturally occurring sequence or a non-naturally occurring sequence. If used for therapeutical purposes, the linker is preferably non-immunogenic in the subject to which the anti-EGFR polypeptide of the invention is administered. One useful group of linker sequences are linkers derived from the hinge region of heavy chain antibodies as described in WO 96/34103 and WO 94/04678. Other examples are poly-alanine linker sequences such as Ala-Ala-Ala. Further preferred examples of linker sequences are Gly/Ser linkers of different length including (gly4ser)3, (gly4ser)4, (gly4ser), (gly3ser), gly3, and (gly3ser2)3.

A “bispecific” polypeptide of the invention is a polypeptide that comprises at least one single-domain antibody directed against a first antigen (i.e. protein S, PS) and at least one further binding site directed against a second antigen (i.e. different from PS), whereas a “trispecific” polypeptide of the invention is a polypeptide that comprises at least one single-domain antibody directed against a first antigen (i.e. PS), at least one further binding site directed against a second antigen (i.e. different from PS) and at least one further binding site directed against a third antigen (i.e. different from both i.e. first and second antigen); etc.

In some embodiments, the further binding site is directed against a serum protein so that the half-lie of the single-domain antibody is increased. Typically, said serum protein is albumin. Typically, the one or more further binding site may comprise one or more parts, fragments or domains of conventional chain antibodies (and in particular human antibodies) and/or of heavy chain antibodies. For example, a single-domain antibody of the invention may be linked to a conventional (typically human) VH or VL optionally via a linker sequence.

In some embodiments, the polypeptides comprise a single-domain antibody of the invention that is linked to an immunoglobulin domain. For example the polypeptides comprise a single-domain antibody of the invention that is linked to an Fc portion (such as a human Fc). Said Fc portion may be useful for increasing the half-life and even the production of the single-domain antibody of the invention. For example the Fc portion can bind to serum proteins and thus increases the half-life on the single-domain antibody. In some embodiments, the at least one single-domain antibody may also be linked to one or more (typically human) CH1, and/or CH2 and/or CH3 domains, optionally via a linker sequence. For instance, a single-domain antibody linked to a suitable CH1 domain could for example be used—together with suitable light chains—to generate antibody fragments/structures analogous to conventional Fab fragments or F(ab′)2 fragments, but in which one or (in case of an F(ab′)2 fragment) one or both of the conventional VH domains have been replaced by a single-domain antibody of the invention. In some embodiments, one or more single-domain antibodies of the invention may be linked (optionally via a suitable linker or hinge region) to one or more constant domains (for example, 2 or 3 constant domains that can be used as part of/to form an Fc portion), to an Fc portion and/or to one or more antibody parts, fragments or domains that confer one or more effector functions to the polypeptide of the invention and/or may confer the ability to bind to one or more Fc receptors. For example, for this purpose, and without being limited thereto, the one or more further amino-acid sequences may comprise one or more CH2 and/or CH3 domains of an antibody, such as from a heavy chain antibody and more typically from a conventional human chain antibody; and/or may form and Fc region, for example from IgG (e.g. from IgG1, IgG2, IgG3 or IgG4), from IgE or from another human Ig such as IgA, IgD or IgM. For example, WO 94/04678 describes heavy chain antibodies comprising a Camelid VHH domain or a humanized derivative thereof (i.e. a single domain antibody), in which the Camelidae CH2 and/or CH3 domain have been replaced by human CH2 and CH3 domains, so as to provide an immunoglobulin that consists of 2 heavy chains each comprising a single domain antibody and human CH2 and CH3 domains (but no CHI domain), which immunoglobulin has the effector function provided by the CH2 and CH3 domains and which immunoglobulin can function without the presence of any light chains.

In some embodiments, the polypeptide is as described in WO2006064136. In particular the polypeptide may consist of i) a first fusion protein wherein the CL constant domain of an antibody is fused by its N-terminal end to the C-terminal end to a single-domain antibody according to the invention (i.e. a single-antibody directed against PS) and ii) a second fusion protein wherein the CH1 constant domain of an antibody is fused by its N-terminal end to the C-terminal end of a single-domain antibody directed against an antigen different from PS. In another particular embodiment, the polypeptide consists of a first fusion protein wherein the CH1 constant domain of an antibody is fused by its N-terminal end to the C-terminal end of a single-domain antibody directed against a an activating trigger molecule on an effector cell (e.g. CD16) and a second fusion protein wherein the CL constant domain of an antibody is fused by its N-terminal end to the C-terminal end to a single-domain antibody of the invention (i.e. PS).

In some embodiment, the polypeptide of the invention is conjugated with further therapeutic agents used to treat thrombotic disorders.

In some embodiments, it is contemplated that the single domain antibody of the invention or the polypeptides of the invention used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications. Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 45 kDa). In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes (see e.g., technologies of established by VectraMed, Plainsboro, N.J.). Such linkers may be used in modifying the polypeptide of the invention described herein for therapeutic delivery.

According to the invention, the single-domain antibodies and polypeptides of the invention may be produced by conventional automated peptide synthesis methods or by recombinant expression. General principles for designing and making proteins are well known to those of skill in the art.

The single-domain antibodies and polypeptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. The single-domain antibodies and polypeptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art.

In another embodiment, the single domain antibody of the invention or the polypeptides of the invention is modified to increase its biological half-life. Various approaches are possible. For example, one or more of the following mutations can be introduced: T252L, T254S, T256F, as described in U.S. Pat. No. 6,277,375 by Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Antibodies with increased half lives and improved binding to the neonatal Fc receptor (FcRn), which is responsible for the transfer of maternal IgGs to the foetus (Guyer et al., J. Immunol. 117:587 (1976) and Kim et al., J. immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311,312, 317, 340, 356, 360, 362, 376, 378, 380, 382, 413, 424, or 434, e.g., substitutions of Fc region residue 434 (U.S. Pat. No. 7,371,826).

Another modification of the single domain antibody of the invention or the polypeptides of the invention herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP0154316 by Nishimura et al. and EP0401384 by Ishikawa et al.

Another modification of the single domain antibody of the invention or the polypeptides of the invention that is contemplated by the invention is a conjugate or a protein fusion of at least the antigen-binding region of the antibody of the invention to serum protein, such as human serum albumin or a fragment thereof to increase half-life of the resulting molecule. Such approach is for example described in Ballance et al. EP0322094. Another possibility is a fusion of at least the antigen-binding region of the antibody of the invention to proteins capable of binding to serum proteins, such human serum albumin to increase half-life of the resulting molecule. Such approach is for example described in Nygren et al., EP 0 486 525.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

### Nucleic Acids, Vectors, Recombinant Host Cells and Uses Thereof

As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), WI38, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the single-domain antibodies and polypeptides of the invention, it would be necessary to employ vectors comprising polynucleotide molecules for encoding the single-domain antibodies and polypeptides of the invention. Methods of preparing such vectors as well as producing host cells transformed with such vectors are well known to those skilled in the art.

Accordingly, a further object of the invention relates to a nucleic acid molecule encoding a single-domain antibody and/or a polypeptide according to the invention.

Typically, said nucleic acid is a DNA or RNA molecule, which may be included in any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

So, a further aspect of the invention relates to a vector comprising a nucleic acid of the invention. Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S. Dak. et al. 1983) of immunoglobulin H chain and the like. Any expression vector for animal cell can be used, so long as a gene encoding the human antibody C region can be inserted and expressed. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

The choice of a suitable expression vector for expression of the peptides or polypeptides of the invention will of course depend upon the specific host cell to be used, and is within the skill of the ordinary artisan.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest (e.g., a single domain antibody). Thus, a promoter nucleotide sequence is operably linked to a given DNA sequence if the promoter nucleotide sequence directs the transcription of the sequence.

A further aspect of the invention relates to a host cell which has been transfected, infected or transformed by a nucleic acid and/or a vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

The nucleic acids of the invention may be used to produce an antibody of the present invention in a suitable expression system. The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like. The present invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

Antibodies of the present invention are suitably separated from the culture medium by conventional immunoglobulin purification procedures such as, for example, protein A-Sepharose, hydroxylapatite chromatography, gel electrophoresis, dialysis, or affinity chromatography.

### Therapeutic Methods and Uses

The single-domain antibodies and polypeptides of the invention enhances the APC-but have no or minor effect on TFPI-cofactor activity of PS. The single-domain antibodies and polypeptides of the invention exerts an in vivo antithrombotic effect in mouse thrombosis model.

Thus, the single-domain antibodies and polypeptides of the invention are particularly suitable for the prevention or treatment of thrombotic disorders in subject in need thereof.

In still another aspect, the invention relates to a single-domain antibody of the present invention and/or polypeptide of the present invention for use as drug.

In particular embodiment, the invention refers to an isolated single-domain antibody of the present invention and/or polypeptide of the present invention for use for treating thrombotic disorders in a subject in need thereof.

In other words, the present invention relates to a method for preventing or treating thrombotic disorders in a subject in need thereof, comprising administering to said subject an effective amount of the single domain antibodies of the present invention and/or polypeptides of the present invention.

As used herein, the term “subject” denotes a mammal. In a preferred embodiment of the invention, a subject according to the invention refers to any subject (preferably human) afflicted with or susceptible to be afflicted with thrombotic disorders.

As used herein, the term “thrombotic disorders”, also known as clotting disorder or thrombophilia, has its general meaning in the art and refers to an inherited or acquired condition that increases the risk of excessive blood clot formation. When a blood vessel is injured, it begins to leak blood either externally or into the tissues. Normal coagulation is important during an injury, as it helps stop a cut from bleeding and starts the healing process. However, if blood tends to clot too much, it is referred to as a hypercoagulable state or thrombophilia. In healthy people, homeostatic balance exists between procoagulant (clotting) forces and anticoagulant and fibrinolytic forces. Numerous genetic, acquired, and environmental factors can tip the balance in favor of coagulation, leading to the pathologic formation of thrombi in veins (eg, deep venous thrombosis [DVT]), arteries (eg, myocardial infarction, ischemic stroke), or cardiac chambers. Thrombi can obstruct blood flow at the site of formation or detach and embolize to block a distant blood vessel (eg, pulmonary embolism, embolic stroke). Acquired conditions are usually a result of surgery, trauma, medications or a medical condition that increases the risk of thrombotic disorders

According to the invention, thrombotic disorders include prothrombin gene mutation, deficiencies of natural proteins that prevent clotting such as antithrombin, protein C and protein S; elevated level of Factor of coagulation such as factor VII, factor IX and XI; factor V Leiden defect; abnormal fibrinolytic system such as hypoplasminogenemia, dysplasminogenemia and elevation in levels of plasminogen activator inhibitor (PAI-1); dysfibrinolysis; central venous catheter placement; restenosis from stents; obesity; hypercoagulability in pregnancy; antiphospholipid antibody syndrome; cancer; homocystinemia; sticky Platelet Syndrome;

pulmonary embolism (PE), Myeloproliferative disorders such as polycythemia vera or essential thrombocytosis; Paroxysmal nocturnal hemoglobinuria (PNH); iatrogenic thromboembolic disorders such as heparin-induced thrombocytopenia (HIT) or thromboembolism induced by haemophilia treatment (emicuzimab, fistusiran, . . . ); Inflammatory bowel syndrome such as ulcerative colitis and Chrohn's disease; acquired immune deficiency syndrome (AIDS); COVID-19; nephrotic syndrome; thrombosis such as acute microthrombosis, distal microvascular thrombosis, deep vein thrombosis (DVT), Paget-Schroetter disease, Budd-Chiari syndrome, portal vein thrombosis, renal vein thrombosis, cerebral venous sinus thrombosis, jugular vein thrombosis, and cavernous sinus thrombosis; limnischemia; sepsis; anemia; sickle-cell disease; cerebral malaria; embolism such as pulmonary embolism and cerebral embolism; and cardiovascular disease such as stroke, myocardial infarction (or heart attack), atrial fibrillation, corony artery disease, congestive heart failure and the placement of prosthetic heart valves.

In some embodiments, the thrombotic disorders are selected from the group consisting of but not limited to in sepsis, sickle-cell anemia; embolism (pulmonary and cerebral) and cardiovascular disease.

In some embodiments, the thrombotic disorders is sepsis or stroke.

In some embodiments, the thrombotic disorders is sickle-cell anemia.

As used herein, the term “sepsis” has its general meaning in the art and represents a serious medical condition that is characterized by a whole-body inflammatory state. In addition to symptoms related to the provoking infection, sepsis is characterized by presence of acute inflammation present throughout the entire body, and is, therefore, frequently associated with fever and elevated white blood cell count (leukocytosis) or low white blood cell count and lower-than-average temperature, and vomiting. In particular, sepsis is defined as a deregulated immune response to infection, translating into life-threatening organs dysfunction, defined by a Sequential Organ Failure Assessment score of 2 points or more. Infection can either be suspected or proven, or a clinical syndrome can be pathognomonic for infection. Septic shock is defined by infection and the need for vasopressors to maintain mean blood pressure >65 mmHg and arterial lactate levels >2 mmol/L.

As used herein the term “stroke” refers to any condition arising from a disruption, decrease, or stoppage of blood or oxygen flow to any part of the brain. In particular, the term “stroke” encompasses, without limitation, ischemic stroke, transient ischemic attack (TIA) and haemorrhagic stroke.

As used herein, the term “embolism” refers to lodging of an embolus, a blockage-causing piece of material, inside a blood vessel. The embolus may be a blood clot (thrombus), a fat globule (fat embolism), a bubble of air or other gas (gas embolism), or foreign material. There are different types of embolism, in the context of the invention, the embolism is caused by a blood clot and is selected from the group consisting of but not limited to: arterial embolism, venous embolism or paradoxical embolism. Typically, the arterial embolism can cause occlusion in any part of the body. It is a major cause of infarction (tissue death from blockage of the blood supply). An embolus lodging in the brain from either the heart or a carotid artery most likely be the cause of a stroke due to ischemia. Typically, the venous embolism refers to an embolus formed in a systemic vein that will always impact the lungs, after passing through the right side of the heart. This will form a pulmonary embolism that will result in a blockage of the main artery of the lung and can be a complication of deep-vein thrombosis. The most common sites of origin of pulmonary emboli are the femoral veins. The deep veins of the calf are the most common sites of actual thrombi. Typically, the venous embolism is pulmonary embolism or cerebral embolism.

As used herein, the term “Cardiovascular disease” also known as or “arteriovascular disease” has a general term used to classify numerous conditions affecting the heart, heart valves, blood, and vasculature of the body and encompasses any disease affecting the heart or blood vessels, including, but not limited to, Metabolic Syndrome, Syndrome X, atherosclerosis, atherothrombosis, coronary artery disease, stable and unstable angina pectoris, stroke, diseases of the aorta and its branches (such as aortic stenosis, thrombosis or aortic aneurysm), peripheral artery disease, peripheral vascular disease, cerebrovascular disease, and including, without limitation, any transiently or permanently ischemic arteriovascular event. Arteriovascular disease as used herein is meant to most commonly refer to the ischemic or pro-ischemic disease, rather than generally to non-ischemic disease. As used herein, “atherosclerosis” and “atherothrombosis” refer to systemic inflammatory disease states associated with complex inflammatory responses to multifaceted vascular pathologies involving inflammatory activation of the endothelium, inflammatory leukocytes as a source of thrombogenic stimuli, smooth muscle cells as a source of procoagulants and amplifier of the inflammatory response during thrombosis, and platelets as mediators of inflammation and thrombosis. Arteries harden and narrow due to build up of a material called “plaque” on their inner walls. As the plaque develops and increases in size, the insides of the arteries get narrower (“stenosis”) and less blood can flow through them. Stenosis or plaque rupture may cause partial or complete occlusion of the affected vasculature. Tissues supplied by the vasculature are thus deprived of their source of oxygenation (ischemia) and cell death (necrosis) can occur. “CAD” or “coronary artery disease” is an arteriovascular disease which occurs when the arteries that supply blood to the heart muscle (the coronary arteries) become atherosclerotic, calcified and/or narrowed. Eventually, blood flow to the heart muscle is reduced, and, because blood carries much-needed oxygen, the heart muscle is not able to receive the amount of oxygen it needs, and often undergoes necrosis. CAD encompasses disease states such as acute coronary syndromes (ACS), myocardial infarction (heart attack), angina (stable and unstable), and atherosclerosis and atherothrombosis that occurs in the blood vessels that supply the heart with oxygen-rich blood. “CVD” or “cerebrovascular disease” is an arteriovascular disease in the blood vessels that feed oxygen-rich blood to the face and brain, such as atherosclerosis and atherothrombosis. This term is often used to describe “hardening” of the carotid arteries, which supply the brain with blood. It is a common comorbid disease with CAD and/or PAD (peripheral artery disease). It is also referred to as an ischemic disease, or a disease that causes a lack of blood flow. CVD encompasses disease states such as cerebrovascular ischemia, acute cerebral infarction, stroke, ischemic stroke, hemorrhagic stroke, aneurysm, mild cognitive impairment (MCI) and transient ischemic attacks (TIA). Ischemic CVD is believed to closely relate to CAD and PAD; non-ischemic CVD may have multiple pathophysiologies.

As used herein, the term “Sickle cell disease” or “SCD” has its general meaning in the art and refers to a hereditary blood disorder in which red blood cells assume an abnormal, rigid, sickle shape. Sickling of erythrocytes decreases the cells' flexibility and results in a risk of various life-threatening complications. The term includes sickle cell anemia, hemoglobin SC disease and Hemoglobin sickle beta-thalessemia. The single gene disorder is characterized by mutant hemoglobin-S (HbS) and chronic intravascular hemolysis. Sickle cell anemia patients often experience episodes of acute pain that are caused by vaso-occlusive crisis (VOC). VOC is the most common complication of sickle cell anemia and a frequent reason for emergency department visits and hospitalization.

As used herein, the term “vaso-occlusive crisis” (VOC) has its general meaning in the art and refers to the the blockage of small blood vessels, which prevents oxygen supply to tissues and causes injury. VOC can be extremely painful and is considered as a medical emergency.

Herein, the inventors demonstrate that the single-domain antibodies and polypeptides of the invention may reduce vaso-occlusive crisis in a mouse model.

In particular embodiment, the invention refers to an isolated single-domain antibody of the present invention and/or polypeptide of the present invention for use for reduces vaso-occlusive crises in a subject in need thereof.

In some embodiment, the subject is afflicted with sickle cell anemia.

In other words, the invention refers to a method for preventing or treating vaso-occlusive crisis (VOC) in a subject in need thereof, comprising administering to said subject an effective amount of the single-domain antibodies of the invention and/or the polypeptide of the invention.

Typically, the single-domain antibodies and polypeptides of the invention and the classical treatment of thrombotic disorders as described above are administered to the subject in a therapeutically effective amount. As used herein, the terms “treatment” or “treating” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, a “therapeutically effective amount” is intended for a minimal amount of active agent which is necessary to impart therapeutic benefit to a patient. For example, a “therapeutically effective amount of the active agent” to a patient is an amount of the active agent that induces, ameliorates or causes an improvement in the pathological symptoms, disease progression, or physical conditions associated with the disease affecting the patient. It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 100 mg/kg of body weight per day,

As used herein the terms “administering” or “administration” refer to the act of injecting or otherwise physically delivering a substance as it exists outside the body (e.g., the nanobody or polypeptide according to the invention) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In another embodiment, the single-domains antibodies or polypeptides according to the invention may be delivered in association with a vector. The single-domains antibodies or drug conjugate of the present invention is included in a suitable vector, such as a plasmid, cosmid, episome, artificial chromosome, phage or a viral vector. So, a further object of the invention relates to a vector comprising a single-domain antibody or drug conjugate of the invention. Typically, the vector is a viral vector, which is an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector. As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g., U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein that allows transduction of cells of human and other species. Typically, the nucleic acid molecule or the vector of the present invention include “control sequences'”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

In a particular embodiment, the single-domain antibodies and polypeptides according to the invention may be used in combination with classical treatment of thrombotic disorders.

Thus, the invention refers to a method for preventing or treating thrombotic disorders in a subject in need thereof, comprising administering to said subject i) an effective amount of the single-domain antibodies and/or polypeptides of the present invention and ii) a classical treatment, as a combined preparation for treating thrombotic disorders.

As used herein, the term “classical treatment of thrombotic disorders” refers to any compound, natural or synthetic, used for the treatment of thrombotic disorders and/or thrombectomy.

According to the invention, compound used for the treatment of thrombotic) may be selected in the group consisting of vitamin K antagonist such as coumarin, warfarin, acenocoumarol, phenprocoumon, atromentin, fluindione and phenindione; heparin and derivative substance such as enoxaparin, dalteparin, nadroparin and tinzaparin; synthetic pentasaccharide inhibitors of factor Xa such as fondaparinux, idraparinux and idrabiotaparinux; directly acting oral anticoagulants such as dabigatran, rivaroxaban, apixaban, edoxaban, and betrixaban, direct thrombin inhibitors such as hirudin, lepirudin, bivalirudin, argatroban and dabigatran; antithrombin protein; batroxobin; hementin; tissue plasminogen activator (tPA); recombinant tissue plasminogen activator (rtPA) such as alteplase, reteplase, urokinase and tenecteplase; streptokinase; anistreplase; platelet aggregation inhibitor such as clopidogrel, prasugrel, ticagrelor, aspirin, triflusal, cangerlor, ticlopidine, cliostazol, vorapaxar, abciximab, eptifibatide, tirofiban, dipyridamole, thromboxane inhibitors and terutroban; platelet GPVI inhibitors such ACT017 and Revacept; inhibitor of P-selectin such as Crizanlizumab activator of protein C such as AB002 (WE Thrombin) and soluble thrombomodulin (BDCA-3); or recombinant activated protein C (APC).

As used herein, the term “thromboectomy” has its general meaning in the art and refers to an interventional procedure of removing a blood clot (thrombus) from a blood vessel. It is commonly performed in the cerebral arteries (interventional neuroradiology). The stent-retriever thromboectomy can be performed with general anesthesia or under conscious sedation in an angiographic room. A system of coaxial catheters is pushed inside the arterial circulation, usually through a percutaneous access to the right femoral artery. A microcatheter is finally positioned beyond the occluded segment and a stent-retriever is deployed to catch the thrombus; finally, the stent is pulled out from the artery, usually under continuous aspiration in the larger catheters. A different technique for thrombectomy in the brain is direct aspiration. It is performed by pushing a large soft aspiration catheter into the occluded vessel and applying direct aspiration to retrieve the thrombus; it can be combined with the stent-retriever technique to achieve higher recanalization rates.

As used herein, the terms “combined treatment”, “combined therapy” or “therapy combination” refer to a treatment that uses more than one medication. The combined therapy may be dual therapy or bi-therapy.

The medications used in the combined treatment according to the invention are administered to the subject simultaneously, separately or sequentially.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different

### Pharmaceutical Compositions and Kits of the Invention

Typically, the single-domain antibodies and polypeptides of the invention (alone or with a vector) may be combined with pharmaceutically acceptable excipients, and optionally sustained-release matrices, such as biodegradable polymers, to form pharmaceutical compositions. Thus, the single-domain antibodies and polypeptides of the invention is administered to the subject in the form of a pharmaceutical composition.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising inhibitors of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The single-domain antibodies and/or polypeptides of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the inhibitors of the invention formulated for parenteral administration, such as intravenous or intramuscular injection, other pharmaceutically acceptable forms include, e.g. tablets or other solids for oral administration; liposomal formulations; time release capsules; and any other form currently used.

Pharmaceutical compositions of the invention may include any further agent which is used in the treatment of thrombotic disorders.

In one embodiment, said additional active agents may be contained in the same composition or administrated separately.

In another embodiment, the pharmaceutical composition of the invention relates to combined preparation for simultaneous, separate or sequential use in the prevention and treatment of thrombotic disorders.

Finally, the invention also provides kits comprising at least one single-domain antibody or polypeptide of the invention. Kits containing the isolated single-domain antibody of the invention and/or polypeptide of the invention find use in therapeutic methods.

The invention will be further illustrated by the following figures and examples. However, these examples and figures should not be interpreted in any way as limiting the scope of the present invention.

### FIGURES

FIG. 1: Schematic representation of the monovalent and bivalent nanobodies used in the present study. The oligonucleotide sequences of monovalent PS003 and KB013 nanobodies were cloned into the pET28-plasmid between the PstI and BstEII restriction sites, to generate nanobodies flanked by an N-terminal His6 tag and a C-terminal HA-tag sequence. Two oligonucleotide sequences of monovalent nanobodies (PS003, KB004, PS004) was fused through a (GGGS)4 linker, synthesized, and cloned into the pET28-plasmid between the PstI and BstEII sites, to generate bivalent nanobodies (PS003biv, KB004biv, PS004biv) that were similarly flanked by an N-terminal His6-tag and a C-terminal HA tag sequence.

FIG. 2: Binding of PS003 to various vitamin K-dependent proteins in ELISA. Recombinant human FIX (FIX), recombinant human FX (FX), plasma-derived protein Z (ProZ), recombinant human Gas6 (Gas6) and recombinant human PS (PS) were immobilized onto ELISA wells, and the binding of 20 nM PS003 was then analyzed.

FIG. 3: Binding of PS003 to PS and Gas6 in ELISA. As PS and Gas6 are highly homologous (47% homology) and both contain a SHBG-like domain, the binding of PS003 to immobilized rhPS and rhGas6 was further analyzed in a direct ELISA. The results demonstrated that PS003 strongly bound to rhPS but not to rhGas6, which confirmed the specificity of PS003 for rhPS.

FIG. 4: Epitope mapping of PS003. rhPS, a recombinant form of the sole SHBG-like domain of PS (rhSHBG), and BSA were immobilized (60 μL at 10 μg/mL in TBS containing 5 mM CaCl₂), and the binding of PS003 was analyzed in a direct ELISA. These results indicated that PS003 was able to bind to rhSHBG, which indicated that the epitope for PS003 was localized within the C-terminal SHBG-like domain of PS. In contrast, PS004 did not bind to rhSHBG (data not shown), suggesting that the epitope for PS004 was localized in the N-terminal part of PS.

FIG. 5: Binding of recombinant human and plasma-derived PS in solution to immobilized PS003 in ELISA. Purified PS003 (60 μL at 10 μg/mL) was immobilized onto ELISA wells and the binding of the two different forms of PS was analyzed. These results indicated that PS003 bound to either recombinant or plasma-derived human PS, and that the binding of PS003 to PS was not restricted to a non-native immobilized form of PS.

FIG. 6: Comparison of PS003 and PS003biv binding to immobilized PS in direct ELISA. rhPS (60 μL at 2.5 μg/mL in TBS containing 5 mM CaCl₂)) was immobilized onto ELISA wells, and the binding of PS003 and PS003biv (0-200 nM) was analyzed with a peroxidase-conjugated polyclonal anti-His6 tag antibody. Three individual experiments were done in simplicate, and the results are expressed as percentage of maximal binding for each nanobody. Binding curves indicated that both PS003 and PS003biv efficiently bound to immobilized rhPS. To further compare the ability of PS003 and PS003biv to bind to PS, the affinity of PS003 and PS003biv for rhPS was estimated as described (Beatty et al. J Immunol Methods 1987), by obtaining similar binding curves on rhPS immobilized at increasing concentrations (0.6, 1.25, 2.5 and 5 μg/mL in TBS containing 5 mM CaCl₂) in three individual experiments done in simplicate. For each nanobody, the dissociation constant (K_{D}) was determined by using a formula based on the Law of Mass Action. Based on this method, the K_{D} of PS003 and PS003biv were 26.8±2.7 nM and 13.8±5.7 nM, respectively, suggesting that PS003biv binds to rhPS with only slightly higher affinity (1.9-fold).

FIG. 7: Epitope mapping of PS003biv and specificity of PS003biv for PS. Recombinant human PS (rhPS), a recombinant form of the PS SHBG-like region alone (rSHBG)), recombinant human Gas6 (rhGas6), or BSA (60 μL at 10 μg/mL in TBS containing 5 mM CaCl₂) were immobilized onto ELISA wells, and the binding of PS003biv (0.5 nM in TBS-0.1% Tween-5 mM CaCl₂)) was analyzed using a peroxidase-conjugated polyclonal anti-His6 tag antibody. The results are expressed as the percentage of the Abs_{450 nm} obtained on rhPS. Three individual experiments were done in simplicate.

The results indicated that PS003biv efficiently bound to rSBHG, and therefore that the epitope of PS003biv is localized within the SHBG-like region of PS. As this region is only found in Gas6, the absence of binding of PS003biv to rhGas6 strongly suggested that PS003biv was specific for PS.

FIG. 8: Enhancing effect of PS003 and PS003biv on the APC-cofactor activity of rhPS in an APTT-based plasma clotting assay (STACLOT® PS, Stago). A. A commercial APTT-based plasma clotting assay (STACLOT® PS, Stago) was used to measure the ability of rhPS (final concentration 5 nM) to act as a cofactor for APC. In this assay, APC prolonged the clotting times of the PS-deficient plasma and 5 nM rhPS further prolonged the clotting times when added together with APC. B. Dose-dependent effects of rhPS (final concentration 0-10 nM) in our APTT-based APC-cofactor activity assay. C. The effects of PS003 and PS003biv were tested on the ability of rhPS (final concentration 6 nM) to enhance the anticoagulant activity of APC. PS003, KB013 (control monovalent nanobody), PS003biv, and KB004biv (control bivalent nanobody) were pre-incubated with rhPS for 15 min at room temperature, and the mixtures of rhPS ±nanobody were added in our assay. The final concentrations of rhPS and the nananobodies were 6 nM and 2 μM, respectively. Experiments were done in triplicate. D. The previous results were expressed as ratios of clotting times in the presence of rhPS (t_{+PS}) to the clotting times in the absence of rhPS (t_{-PS}). Unpaired Student's t-test was used as a statistical test.

The results demonstrated that both PS003 and PS003biv enhanced the APC-cofactor activity of rhPS in our plasma-based assay, and that the enhancing effect of PS003biv on the APC-cofactor activity of rhPS appeared higher than that of PS003.

FIG. 9: Effects of PS003 and PS003biv on the APC-cofactor activity of PS in an in vitro FVa inactivation assay. The capacity of PS003 and PS003biv to enhance the APC-cofactor activity of rhPS was evaluated in an in vitro assay measuring the specific proteolytic inactivation of FVa by APC, in the presence of rhPS, using purified proteins. A. The slopes were determined for each rhPS concentration in the FVa inactivation mixture, and a value of FVa activity was expressed as the ratio between the slope obtained in the presence of rhPS and the slope obtained in the absence of rhPS. Three experiments were done in simplicate. B. The residual FVa activity was determined for each condition using a prothrombinase assay as previously described and compared to the FVa activity obtained when rhPS was pre-incubated in the absence of nanobodies or antibodies (TBS). Three experiments were done in simplicate and Unpaired Student's t-test was used as a statistical test (*** P<0.001).

FIG. 10: Effects of PS003 and PS003biv on the TFPI-cofactor activity of rhPS. A. An in vitro assay has been developed to assess the ability of rhPS to enhance the direct inhibition of FXa by TFPIα. A. Recombinant human full-length TFPIα expressed in E. coli was used at a final concentration of 5 nM to inhibit the amidolytic activity of FXa. B. The ability of rhPS to enhance the inhibitory activity of TFPIα was studied by pre-incubating for 15 min rhPS at room temperature with a blocking rabbit polyclonal anti-PS antibody (α-PS) (DAKO, final concentration 0.5 μM), or with rabbit IgG (DAKO, final concentration 0.5 μM. C. The ability of rhPS to enhance the inhibitory activity of TFPIα was evaluated when rhPS was pre-incubated for 15 min at room temperature with PS003 and PS003biv, or their respective monovalent (KB013) and bivalent (KB004biv) control nanobodies (final concentration 10 μM). The results were expressed as percentage of the TFPIα-cofactor activity of rhPS in the absence of nanobodies (TBS), and three experiments were done in simplicate, and Unpaired Student's t-test was used as a statistical test.

FIG. 11: Comparison of PS003biv and PS004biv binding to immobilized recombinant murine PS in direct ELISA. PS004biv is an in-house anti-human PS nanobody generated from a monovalent nanobody (PS004) identified after a selection on rhPS immobilized onto ELISA wells. PS004biv strongly binds to rhPS in direct ELISA but, in contrast to PS003biv, its epitope is localized within the N-terminal part of PS and not within the SHBG-like domain of PS (data not shown). The binding of PS003biv and PS004biv to immobilized rmPS was analyzed by ELISA. The results demonstrated that PS003biv, but not PS004biv, bound to rmPS. This indicated that PS004biv could be used as a control bivalent nanobody, together with PS003biv, in our in vivo FeCl₃-induced thrombosis model.

FIG. 12: In vivo antithrombotic effects of PS003biv in a mouse FeCl₃-induced thrombosis model. FeCl₃-injury was induced in 4- to 5-week-old C57BL6/JRccHsd male mice, essentially as previously described (Aymé et al. 2017; Adam et al. 2010). To facilitate visualization of thrombus formation, platelets of anesthetized mice were fluorescently labeled in vivo by intravenous injection of rhodamine 6G (3.3 mg/kg, i.e. 2.5 μL/g of rhodamine 6G at 1 mg/mL in 0.9% NaCl) into the retro-orbital plexus. PS003biv (10 mg/kg), PS004biv (10 mg/kg), or the same volumes of TBS buffer (Ctl), were diluted in 0.9% NaCl and were administered simultaneously. Alternatively, 200 UI/kg low-molecular-weight heparin (LMWH, Lovenox) was injected subcutaneously after the intravenous injection of rhodamine 6G. The labelled platelets were left to circulate for 10 min, and after a topical deposition of FeCl₃ solution (10% in water) on the mesenteric vessels, thrombus growth was monitored in real-time with an inverted epifluorescent microscope (×10). One single venule and one single arteriole were analyzed for each mouse. Statistical analysis was assessed via Kruskal Wallis and Dunn's test. A. The control bivalent anti-VWF (KB004biv) used in our APC-cofactor activity assay could not be used in our FeCl₃-induced thrombosis model as treatment of mice with this nanobody resulted in delayed occlusion times in the venule and arteriole of one mouse. Thus, we used a control bivalent anti-PS nanobody (PS004biv) that is not able to bind to recombinant murine PS. Our thrombosis model was sensitive to an anticoagulant drug as treatment of mice with LMWH (200 UI/kg, SC) resulted in delayed occlusion times in venules and arterioles (n=6 mice). Whereas treatment with PS004biv (n=6 mice) had no effect on occlusion times, treatment with PS003biv led to significantly delayed occlusion times in venules (n=10 mice). A similar trend was observed in the arterioles (n=9 mice) of mice treated with PS003biv but no statistic differences could be demonstrated. B. In the mesenteric vessels of mice administered with PS003biv, thrombi were found to be less stable with a high rate of embolization, as compared with the thrombi formed in the mesenteric vessels of mice not administered with nanobodies (not shown) or administered with control PS004biv nanobody.

FIG. 13: Effects of PS003biv on physiological hemostasis in a mouse tail-clip bleeding model. Anesthetized C57/BL6 mice were intravenously injected with PS003biv (10 mg/kg) or subcutaneously injected with low-molecular-weight heparin (LMWH) (Lovenox, 200 UI/kg). Bleeding time was defined as the first cessation of bleeding. Blood was also collected during 20 minutes to quantify total blood loss volumes. Each bar represents the mean obtained from several mice evaluated. Ordinary one-way ANOVA was used as a statistical test of variance with Tukey's multiple comparison test.

### EXAMPLE 1

Material & Methods

Selection of the PS003 Nanobody by Phage-Display

Anti-PS nanobodies were identified essentially as previously described for anti-VWF nanobodies (Aymé et al. 2017). Briefly, immunization of a single llama (L. glama) with recombinant human PS (rhPS) was outsourced to the Centre de Recherche en Cancérologie (Université Aix-Marseille, Marseille, France). Blood was collected for the isolation of peripheral blood lymphocyte, and lymphocyte total mRNA was used for the construction of a single-domain antibody (sdAb)-library. Briefly, total mRNA was used for the synthesis of cDNA via reverse transcriptase using a CH2′ primer. sdAb-coding DNA fragments were obtained from the cDNA by a nested-PCR reaction, and fragments were subsequently cloned into the pHEN6 phagemid vector. The ligated material was used to transform competent TG1 E. coli cells (ThermoFischer Scientific) allowing the creation of a library of >107 transformants. Phages exposing each sdAbs were rescued by infection of a culture of this library with M13K07-helper phages, and phage particles were incubated with Dynabeads M-450 epoxy beads coated with purified rhPS (1 mg/mL) for 1 h at room temperature in 50 mM Tris, 150 mM NaCl pH 7.4 (TBS Buffer) containing 2% BSA and 5 mM CaCl2. Magnetic beads were washed nine times with using TBS containing 0.1% Tween-20 and 5 mM CaCl2), and twice with TBS containing 5 mM CaCl2. Captured phages were eluted by incubation with 500 μL of 1 mg/mL trypsin in TBS, for 30 min at room temperature. Eluted phages (500 μL) were diluted in 500 μL of TBS, and 5 μL of eluted phages were serially diluted to infect TG1 E. coli cells, and plated to evaluate the PS-specific enrichment. The remainder of the eluted phage solution was amplified after rescue using M13K07-helper phages to perform a new round of enrichment. Two consecutive rounds of enrichment were performed. After the second round of enrichment, 5 μL of eluted phages were serially diluted to infect TG1 E. coli cells, and plated to obtain single ampicillin-resistant colonies. To isolate genuine PS-specific nanobodies, these TG1-clones were grown overnight in 96-deep-well culture plates in 0.5 mL 2YT-medium and nanobody expression was induced with 1 mM IPTG. Periplasmic extracts containing the nanobodies were prepared as described and tested for binding to immobilized rhPS or BSA in a direct ELISA. This allowed the identification of a strong and specific PS binder, named PS003.

Construction of PS003 and PS003biv Nanobodies

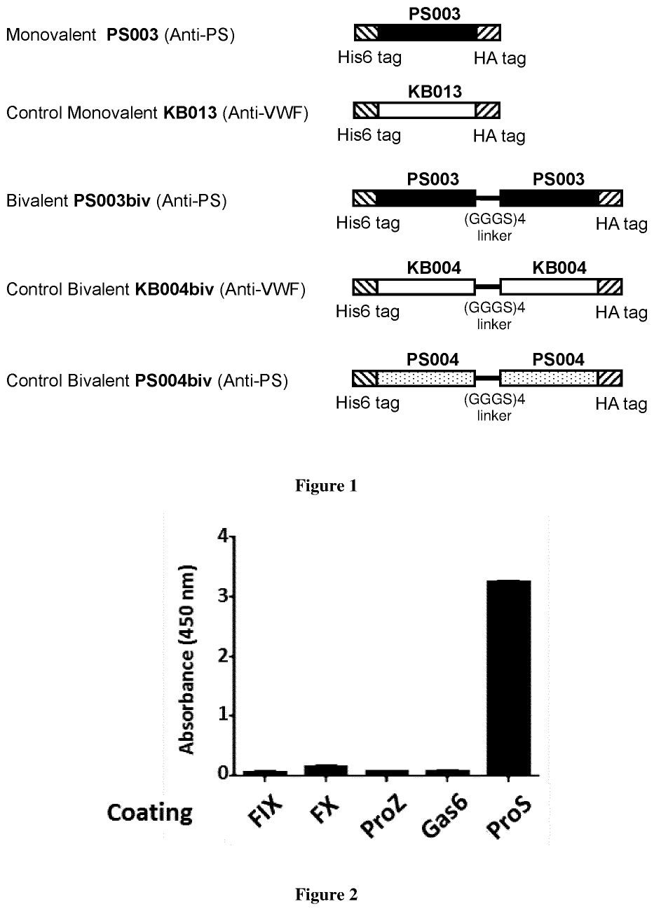

To allow intracytoplasmic bacterial expression, the cDNA sequence of PS003 was cloned into the pET28-plasmid between the 5′ PstI and 3′ BstEII restriction sites. In this pET28 format, the protein sequence of PS003 is flanked by an N-terminal His6-tag and a C-terminal haemagglutinin (HA)-tag to facilitate purification and detection (FIG. 1). To potentially increase the affinity and activity of PS003, a bivalent form of PS003biv (named PS003biv) was constructed by fusing two cDNA sequences of PS003 through a flexible (GGGS)4 linker (FIG. 1). The cDNA sequence of PS003biv was synthetized (ProteoGenix, France) and cloned into the pET28-plasmid between the PstI and BstEII restriction sites. A monovalent anti-VWF nanobody (KB013) and a bivalent anti-VWF nanobody (KB004biv) were used as controls in our in vitro functional assays. The cDNA sequences of these nanobodies were cloned into the pET28-plasmid, as previously described for PS003 and PS003biv. For our in vivo FeCl₃-induced thrombosis model, a bivalent anti-PS nanobody was used as a control. This anti-PS nanobody, named PS004biv, was constructed from a monovalent nanobody (PS004) which was identified by selecting phage particles on PS that was immobilized onto ELISA wells. Three rounds of enrichment were used to identify a monovalent nanobody (PS004) which strongly and specifically binds to immobilized PS in ELISA. The cDNA sequence of PS004biv was synthetized and cloned into the pET28-plasmid. All the nanobodies used in the present study are flanked by an N-terminal His6-tag and a C-terminal HA-tag, and all the bivalent nanobodies are linked by a (GGGS)4 linker.

Expression and Purification of Nanobodies The plasmids encoding monovalent and bivalent nanobodies were used to transform competent T7 SHuffle E. coli cells (New England Biolabs). For each nanobody, a single kanamycin-resistant colony was cultured at 30° C. in LB medium containing 30 μg/mL kanamycin until 0.4<OD_{600 nm}<0.6. The cytoplasmic expression of nanobodies was then induced by the addition of 0.1 mM IPTG, and nanobodies were produced for 16 h at 20° C. Bacterial pellets were resuspended in 50 mM NaH₂PO₄, 0.3 M NaCl pH 7.4 containing 10 μg/mL lysozyme (Sigma) and 25 U/mL benzonase (Sigma), and supplemented with SigmaFAST protease inhibitors (Sigma). The suspension was sonicated and centrifuged at 12000 rpm for 30 min at 4° C. Lysates were frozen at −20° C.

Monovalent nanobodies were purified through immobilized metal ion chromatography (IMAC). Briefly, lysates were thawed at 37° C. and centrifuged at 4700 rpm for 30 min at 20° C. Supernatants were loaded at 1 mL/min on a HiTrap TALON Crude column (GE Healthcare) pre-equilibrated in 50 mM NaH₂PO₄, 0.3 M NaCl pH 7.4. The column was washed with >20 column volumes of 50 mM NaH₂PO₄, 0.3 M NaCl pH 7.4, and washed with >20 column volumes of 50 mM NaH₂PO₄, 0.3 M NaCl pH 7.4 containing 10 mM imidazole. The bound nanobodies were eluted with 50 mM NaH₂PO₄, 0.3 M NaCl pH 7.4 containing 150 mM imidazole, and fractions (1 mL) were collected. The protein content in each fraction was determined by measuring the OD_{280 nm}, and the fractions of interest were pooled and dialyzed against 50 mM Tris, 150 mM NaCl, pH 7.4 (TBS buffer). The dialysate was eventually concentrated on an Amicon® Ultra-15 centrifugal filter device (3 kDa cut-off) (Merck Millipore).

Bivalent nanobodies were purified through Protein A affinity chromatography. Briefly, lysates were thawed at 37° C. and centrifuged at 4700 rpm for 30 min at 20° C. Supernatants were loaded at 0.5-1 mL/min on a HiTrap Protein A Fast Flow column (GE Healthcare) pre-equilibrated in 50 mM NaH₂PO₄, 0.3 M NaCl pH 7.4. The column was washed with >20 column volumes with 50 mM NaH₂PO₄, 0.3 M NaCl pH 7.4, and the bound nanobody was eluted with 0.1 M Glycine pH 2.7. Fractions (1 mL) were collected in tubes containing 100 μL of 1M Tris·HCl pH 8.5. The protein content in each fraction was determined by measuring the OD_{280 nm}, and the fractions of interest were pooled and dialyzed against TBS buffer. The dialysate was eventually concentrated on an Amicon® Ultra-15 centrifugal filter device (3 kDa cut-off) (Merck Millipore).

Epitope Mapping of PS003

A recombinant form of the PS SHBG-like domain alone (rSHBG) was previously expressed and purified (Saposnik et al. 2003). BSA, purified rhPS and purified rSHBG (60 μL at 8 μg/mL in TBS containing 5 mM CaCl₂)) were immobilized onto 96-well NUNC Maxisorp plates for 16 h at 4° C. Wells were washed with 3×200 μL of washing buffer (TBS containing 5 mM CaCl₂ and 0.1% Tween-20) and wells were blocked for 1 h at room temperature with TBS containing 5 mM CaCl₂ and 5% BSA. Wells were washed with 3×200 μL of washing buffer and a fixed concentration of PS003 (2 nM in TBS containing 5 mM CaCl₂ and 1% BSA, 50 μL/well) was incubated for 1 h at 37° C. Wells were washed with 3×200 μL of washing buffer, and a peroxidase-conjugated polyclonal anti-HA tag antibody (Abcam, 1 μg/mL in TBS containing 5 mM CaCl₂ and 1% BSA, 50 μL/well) was incubated for 1 h at room temperature. Wells were washed with 3×200 μL of washing buffer and 50 μL of TMB was added. Reactions were stopped by the addition of 50 μL of 2M H₂SO₄. The results demonstrated that the epitope for PS003 was localized within the SHBG-like domain of PS (FIG. 4). Furthermore, the epitope for PS003 was not found within the Gas6 SHBG-like domain (FIG. 4). Amino-acid residues that are not conserved between the PS and Gas6 SHBG-like domains thus appear as candidates for mediating the interaction between PS003 and PS.

Results

Specificity of PS003 for rhPS

To gain insights into the specificity of PS003, we tested the ability of purified PS003 to bind to PS and to various vitamin K-dependent proteins that contain homologous domains (Gla and EGF-like domains). Recombinant human PS (rhPS), recombinant human FIX (BeneFIX, Pfizer), recombinant human FX (Haematologic technologies), plasma-derived protein Z (Hyphen BioMed), and recombinant human Gas6 (rhGas6) (Clauser et al. 2012) (60 μL at 10 μg/mL in TBS containing 5 mM CaCl₂) were immobilized onto 96-well NUNC Maxisorp plates for 16 h at 4° C. Wells were washed with 3×200 μL of washing buffer (TBS containing 0.1% Tween-20 and 5 mM CaCl₂) and wells were blocked for 1 h at room temperature with TBS containing 5 mM CaCl₂) and 5% BSA. Wells were washed with 3×200 μL of washing buffer and a fixed concentration (20 nM in TBS containing 5 mM CaCl₂ and 1% BSA, 50 μL/well) of purified PS003 was incubated for 1 h at room temperature. Wells were washed with 3×200 μL of washing buffer, and a peroxidase-conjugated polyclonal anti-HA tag antibody (Abcam, 1 g/mL in TBS containing 5 mM CaCl₂ and 1% BSA, 50 μL/well) was incubated for 1 h at room temperature. Wells were washed with 3×200 μL of washing buffer and 50 μL of TMB was added. Reactions were stopped by the addition of 50 μL of 2M H₂SO₄. The results suggested that PS003 strongly bound only to rhPS (FIG. 2) and that PS003 was thus rather specific for PS.

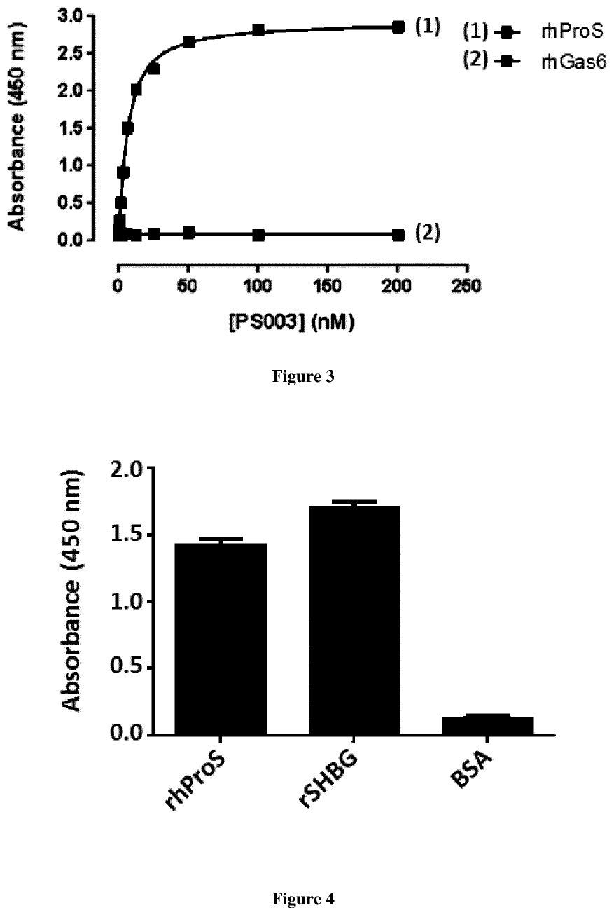

Vitamin K-dependent Gas6 is highly homologous to PS (47% overall homology) and, in contrast to other vitamin K-dependent proteins, also contains a SHBG-like domain. To further confirm the specificity of PS003 to PS, rhPS and rh Gas6 (60 μL, at 10 μg/mL in TBS containing 5 mM CaCl₂) were immobilized onto 96-well NUNC Maxisorp plates for 16 h at 4° C. Wells were washed with 3×200 μL of washing buffer (TBS containing 5 mM CaCl₂ and 0.1% Tween-20) and wells were blocked for 1 h at room temperature in TBS containing 5 mM CaCl₂ and 5% BSA. Wells were washed with 3×200 μL of washing buffer and increasing concentrations of PS003 (0-200 nM in TBS containing 5 mM CaCl₂ and 1% BSA, 50 μL/well) was incubated for 1 h at room temperature. Wells were washed with 3×200 μL of washing buffer, and a peroxidase-conjugated polyclonal anti-HA tag antibody (Abcam, 1 μg/mL in TBS containing 5 mM CaCl₂ and 1% BSA, 50 μL/well) was incubated for 1 h at room temperature. Wells were washed with 3×200 μL of washing buffer and 50 μL of TMB was added. Reactions were stopped by the addition of 50 μL of 2M H₂SO₄. The results demonstrated that PS003 strongly and dose-dependently bound to rhPS but not to rhGas6 (FIG. 3), which confirmed the specificity of PS003 for rhPS.

Binding of Recombinant and Plasma-Derived PS to Immobilized PS003 in Sandwich ELISA

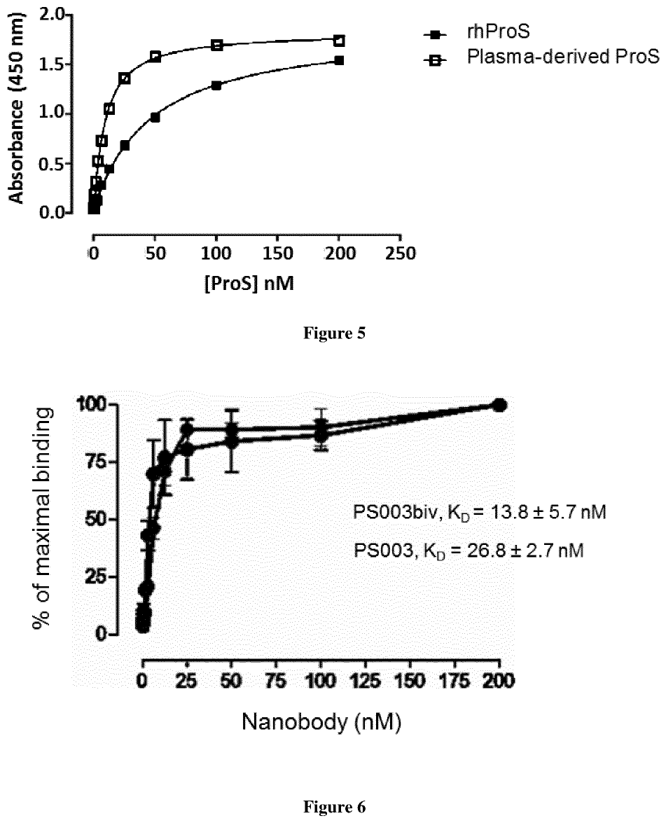

In our phage-display strategy, PS003 was selected on an immobilized form of rhPS covalently coupled to magnetic beads. In addition, PS003 was found to strongly bind to rhPS immobilized onto ELISA wells. To rule out that PS003 only recognizes a non-native immobilized form of rhPS, the binding of rhPS in solution to immobilized PS003 was analyzed by a sandwich ELISA. In addition, as PS003 was selected on a recombinant form of PS, we analyzed the binding of a plasma-derived form of PS in solution to PS003 in the same sandwich ELISA. Briefly, rhPS and purified plasma-derived human PS (Haematologic Technologies) (60 μL at 10 μg/mL in TBS containing 5 mM CaCl₂) were immobilized onto 96-well NUNC Maxisorp plates for 16 h at 4° C. Wells were washed with 3×200 4 of washing buffer (TBS containing 5 mM CaCl₂) and 0.1% Tween-20) and wells were blocked for 1 h at room temperature with TBS containing 5 mM CaCl₂ and 5% BSA. Wells were washed with 3×200 μL of washing buffer and increasing concentrations (0-200 nM in TBS containing 5 mM CaCl₂) and 1% BSA, 50 μL/well) of PS003 was incubated for 1 h at room temperature. Wells were washed with 3×200 μL of washing buffer, and a peroxidase-conjugated polyclonal anti-HA tag antibody (Abcam, 2 μg/mL in TBS containing 5 mM CaCl₂ and 1% BSA, 50 μL/well) was incubated for 1 h at room temperature. Wells were washed with 3×200 4 of washing buffer and 50 μL of TMB was added. Reactions were stopped by the addition of 50 μL of 2M H₂SO₄. The results indicated that PS003 bound to either recombinant or plasma-derived human PS (FIG. 5), and that the binding of PS003 to PS was not restricted to a non-native immobilized form of PS.

Comparison of PS003 and PS003biv Binding to Immobilized PS in ELISA

Recombinant human PS (rhPS) (60 μL at 2.5 μg/mL in TBS containing 5 mM CaCl₂)) was immobilized onto 96-well NUNC Maxisorp plates, for 16 h at 4° C. Wells were washed with 3×200 μL of washing buffer (TBS containing 5 mM CaCl₂) and 0.1% Tween-20) and wells were blocked for 1 h at room temperature with TBS containing 5 mM CaCl₂) and 5% BSA. Wells were washed with 3×200 μL of washing buffer and increasing concentrations of PS003 and PS003biv (0-200 nM in TBS containing 5 mM CaCl₂ and 1% BSA, 50 μL/well) were incubated for 1 h at room temperature. Wells were washed with 3×200 μL of washing buffer, and a peroxidase-conjugated polyclonal anti-His6 tag antibody (Abcam, 1 μg/mL in TBS containing 5 mM CaCl₂) and 1% BSA, 50 μL/well) was incubated for 1 h at room temperature to detect the bound nanobodies. Wells were washed with 3×200 4 of washing buffer and 50 μL of TMB was added. Reactions were stopped by the addition of 50 μL of 2M H₂SO₄. For each nanobody, three individual experiments were done in simplicate, and the results are expressed as percentage of maximal binding for each nanobody. Binding curves indicated that both PS003 and PS003biv efficiently bound to immobilized rhPS (FIG. 6).

To further compare the ability of PS003 and PS003biv to bind to PS, the affinity of PS003 and PS003biv for rhPS was estimated as described (Beatty et al. J Immunol Methods 1987), by obtaining binding curves on rhPS immobilized at increasing concentrations (0.6, 1.25, 2.5 and 5 μg/mL in TBS containing 5 mM CaCl₂) in three individual experiments done in simplicate. For each nanobody, the dissociation constant (K_{D}) was determined by using a formula based on the Law of Mass Action.

Based on this method, the K_{D} of PS003 and PS003biv were 26.8±2.7 nM and 13.8±5.7 nM, respectively, suggesting that PS003biv bound to rhPS with only slightly higher affinity (1.9-fold).

Epitope mapping of PS003biv and specificity of PS003biv for PS rhPS, a recombinant form of the PS SHBG-like region alone (rSHBG) (Saposnik et al. 2003), recombinant human Gas6 (rhGas6), or BSA (60 μL at 10 μg/mL in 50 mM Tris, 150 mM NaCl, pH 7.4 (TBS) containing 5 mM CaCl₂) were immobilized onto 96-well NUNC Maxisorp plates, for 16 h at 4° C. Wells were washed with 3×200 μL of washing buffer (TBS containing 5 mM CaCl₂ and 0.1% Tween-20) and wells were blocked for 1 h at room temperature with TBS containing 5 mM CaCl₂ and 5% BSA. Wells were washed with 3×200 μL of washing buffer and 0.5 nM PS003biv (in TBS containing 5 mM CaCl₂, 0.1% Tween-20 and 2% BSA, 50 μL/well) were incubated for 1 h at room temperature. Wells were washed with 3×200 μL of washing buffer, and a peroxidase-conjugated polyclonal anti-His6 tag antibody (Abcam, 1 μg/mL in in TBS containing 5 mM CaCl₂, 0.1% Tween-20 and 2% BSA, 50 μL/well) was incubated for 1 h at room temperature to detect the bound nanobodies. Wells were washed with 3×200 μL of washing buffer and 50 μL of TMB was added. Reactions were stopped by the addition of 50 μL of 2M H₂SO₄. The results are expressed as the percentage of the Abs_{450 nm} obtained on rhPS. Three individual experiments were done in simplicate.

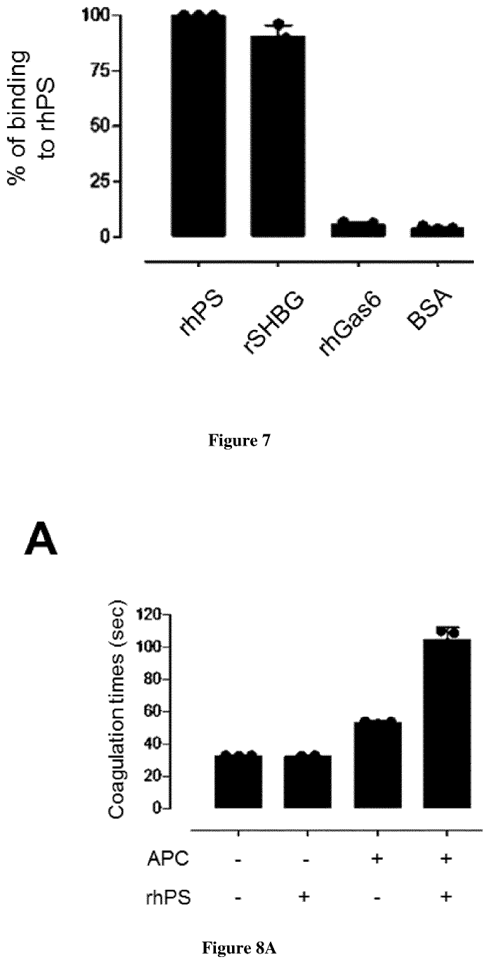

The results indicated that PS003biv efficiently bound to rSBHG (FIG. 7) and that the epitope of PS003biv was localized within the SHBG-like region of PS. As this region is only found in Gas6, the absence of binding of PS003biv to rhGas6 (FIG. 7) strongly suggested that PS003biv was specific for PS.

Enhancing Effects of PS003 and PS003biv on the APC-Cofactor Activity of PS in an APTT-Based Plasma Clotting Assay

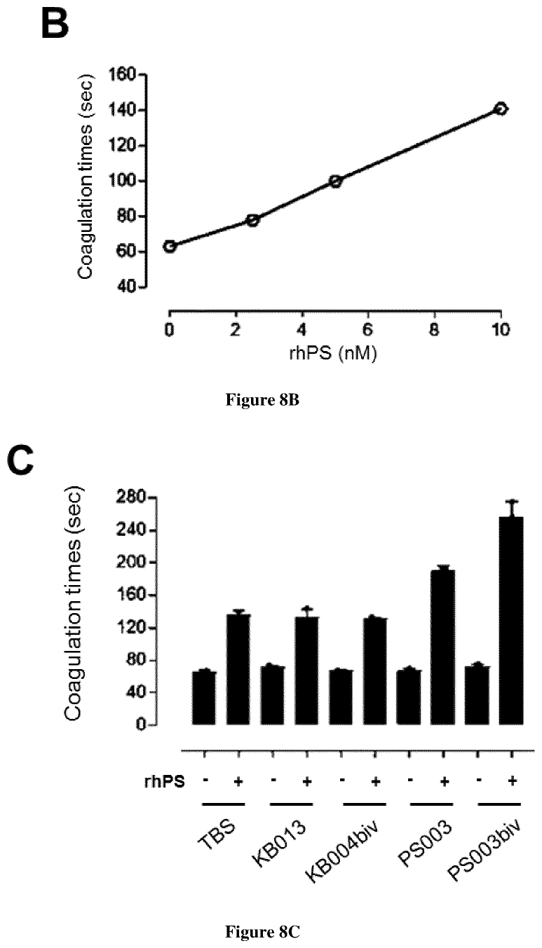

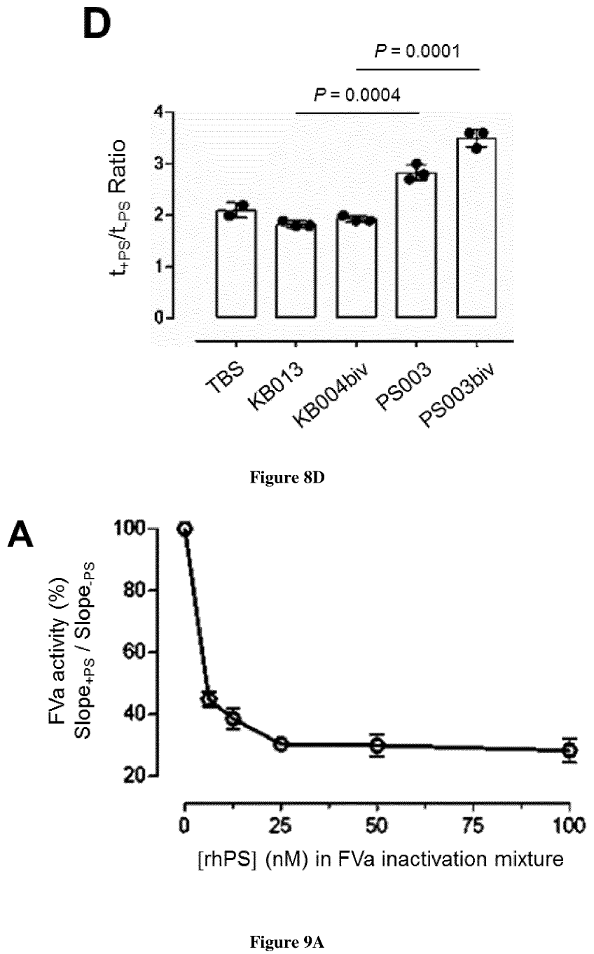

We used a commercial APTT-based plasma clotting assay (STACLOT® PS, Stago) on a KC4 Coagulometer (Stago) to measure the ability of rhPS to act as a cofactor for APC in the inactivation of FVa and FVIIIa. Briefly, 25 μL of rhPS diluted in TBS containing 0.1% BSA were added to 25 μL of a PS-deficient plasma (R1 Reagent) together with APC (R2 Reagent, 4) and bovine FVa (Reagent R3, 25 μL). After a 2-min incubation at 37° C., clotting was triggered by the addition of 25 μL of 50 mM CaCl₂. In this assay, APC prolonged the clotting times of the PS-deficient plasma, and rhPS (final concentration 5 nM) further prolonged, in a dose-dependent manner, the clotting times when rhPS was added together with APC (FIG. 8A). This prolongation reflects the APC-cofactor activity of rhPS. In this assay, rhPS does not prolong the clotting times in the absence of APC (FIG. 8A). In this assay, the ability of rhPS to enhance the anticoagulant activity of APC was abolished by a polyclonal anti-PS antibody (DAKO, A0384), which had largely been described to potently block the APC-cofactor activity of rhPS (data not shown), further demonstrating that this assay is very dependent on the presence of rhPS.

A dose-response curve demonstrated that rhPS dose-dependently prolongs the clotting times in the presence of a fixed concentration of APC (FIG. 8B). An intermediate concentration of rhPS (6 nM) resulting in a ratio t_{+PS}/t_{-PS} of ˜2 was chosen to be able to detect any inhibitory or stimulatory effects of nanobodies.

We then tested the effects of PS003 and PS003biv on the ability of rhPS (6 nM) to enhance the anticoagulant activity of APC. PS003, KB013, PS003biv, and KB004biv were pre-incubated at 10 μM with rhPS (30 nM) in TBS containing 0.1% BSA, for 15 min at room temperature. The mixture of rhPS ±nanobody (25 μL) was added to 25 μL of a PS-deficient plasma (R1 Reagent) together with APC (R2 Reagent, 25 μL) and bovine FVa (Reagent R3, 25 μL). After a 2-min incubation at 37° C., clotting was triggered by the addition of 25 μL of 50 mM CaCl₂. The final concentrations of rhPS and the nananobodies were 6 nM and 2 μM, respectively. Experiments were done in triplicate.

In the presence of rhPS and APC, the clotting times were prolonged by approximately 2-fold in the absence (TBS) and in the presence of monovalent (KB013) and bivalent (KB004biv) nanobodies (final concentration 2 μM), which reflects the normal APC-cofactor of rhPS (FIG. 8C). In contrast, clotting times were further prolonged by 2.8- and 3.6-fold in the presence of PS003 and PS003biv respectively (final concentration 2 μM), thus reflecting a surprising enhancing effect of PS003 and PS003biv on the APC-cofactor activity of rhPS, as compared to their respective control nanobodies (FIG. 8C). In addition, the enhancing effect of PS003biv on the APC-cofactor activity of rhPS appeared higher than that of PS003.

The previous results were expressed as ratios of clotting times in the presence of rhPS (t_{+PS}) to the clotting times in the absence of rhPS (t_{-PS}) (FIG. 8D). Unpaired Student's t-test was used as a statistical test.

Effects of PS003 and PS003biv on the APC-Cofactor Activity of PS in an In Vitro FVa Inactivation Assay

The capacity of PS003 and PS003biv to enhance the APC-cofactor activity of rhPS was evaluated in an in vitro assay measuring the specific proteolytic inactivation of FVa by APC, in the presence of rhPS, using purified proteins. Plasma-derived human FVa (Haematologic Technologies, 80 nM) was inactivated for 20 min with plasma-derived human APC (Haematologic Technologies, 0.5 nM), in the presence of 25 μM PC/PE/PS phospholipid vesicles and increasing concentration of rhPS (0-100 nM) in 50 mM Tris, 150 mM NaCl, pH 7.4 (TBS) containing 5 mM CaCl₂, 0.2% PEG and 0.2% BSA (“FVa inactivation mixture”). The reaction was stopped by diluting the FVa inactivation mixture (1:10) in TBS containing 5 mM CaCl₂, 0.2% PEG and 0.2% BSA. The residual FVa activity was then measured in a prothrombinase assay with plasma-derived human prothrombin (Haematologic Technologies, 200 nM) and FXa (Enzyme Research Laboratories, 200 nM), in in TBS containing 5 mM CaCl₂, 0.2% PEG and 0.2% BSA and 50 μM PC/PS/PE phospholipid vesicles. The amidolytic activity of thrombin was followed using a chromogenic substrate (pNAPEP0238, 200 μM) in TBS containing 10 mM EDTA, 0.2% PEG and 0.2% BSA, and the slope of progress curves were calculated. The slopes were determined for each rhPS concentration in the FVa inactivation mixture, and a value of FVa activity was expressed as the ratio between the slope obtained in the presence of rhPS and the slope obtained in the absence of rhPS. Three experiments were done in simplicate.

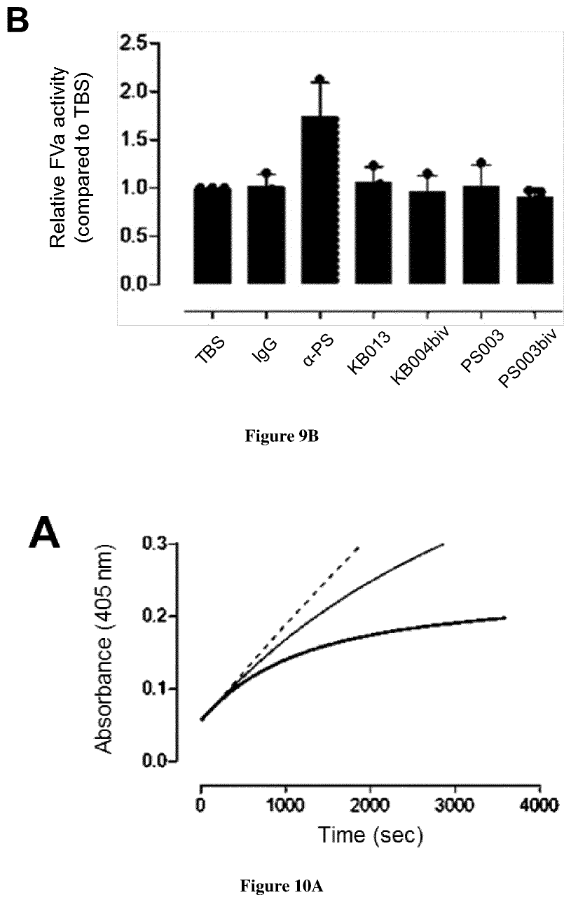

The results demonstrated that rhPS dose-dependently and very efficiently enhanced the ability of APC to inactivate FVa (FIG. 9A), and a concentration of 6 nM rhPS was chosen to evaluate the effects of PS003 and PS003biv. To verify that our assay was dependent on the presence of rhPS, we also used a polyclonal anti-PS antibody (DAKO, A0384) which has largely been described to potently block the APC-cofactor activity of rhPS. Thus, 80 nM FVa was inactivated for 20 min with 0.5 nM APC, 25 μM PC/PS/PE phospholipids vesicles, and 6 nM rhPS was pre-incubated or not (TBS) for 15 min with nanobodies (PS003, PS003biv, control monovalent nanobody KB013, and control bivalent nanobody KB004biv; final concentration 10 μM), rabbit polyclonal anti-PS antibody (α-PS, DAKO; final concentration 0.5 μM), and rabbit IgG (DAKO; final concentration 0.5 μM). The residual FVa activity was determined for each condition using a prothrombinase assay as previously described and compared to the FVa activity obtained when rhPS was pre-incubated in the absence of nanobodies or antibodies (TBS). Three experiments were done in simplicate and Unpaired Student's t-test was used as a statistical test (***P<0.001).

The results demonstrated that, in this APC-cofactor activity assay, the blocking anti-PS antibody (α-PS, DAKO) effectively inhibited the APC-cofactor activity of rhPS (FIG. 9B). In contrast to what was observed in an APTT-based APC-cofactor activity assay, PS003 and PS003biv had no potentiating effect on the APC-cofactor activity of rhPS in this “reductionist” FVa inactivation assay (FIG. 9B).

Effects of PS003 and PS003biv in an In Vitro TFPIα-Cofactor Activity Assay of PS

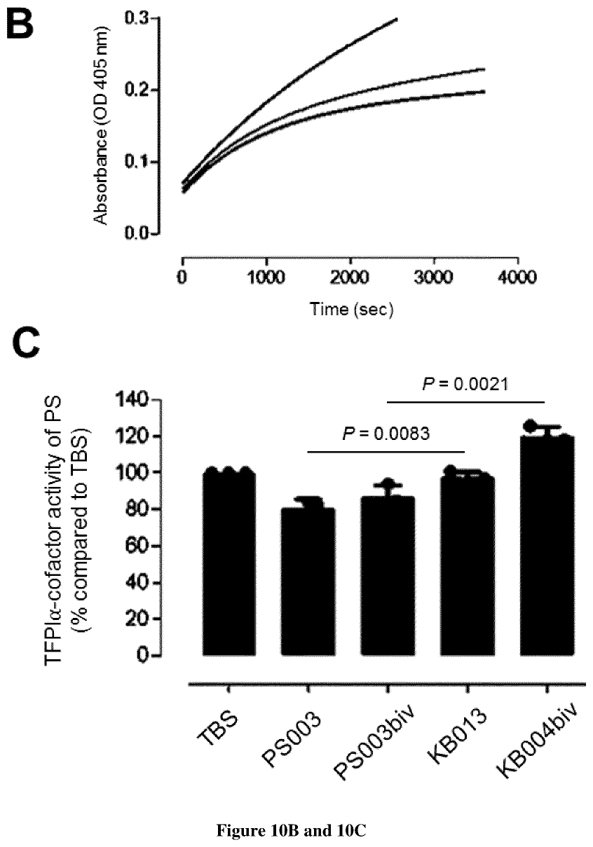

An in vitro assay has been developed to assess the ability of rhPS to enhance the direct inhibition of FXa by TFPIα. The amidolytic activity of plasma-derived human FXa (Enzyme Research Laboratories, final concentration 0.2 nM) towards a chromogenic substrate specific for FXa (pNAPEP, Cryopep, 400 μM) in a final volume of 100 μL of TBS containing 10 mM CaCl₂, 0.2% PEG, 0.2% BSA and 25 μM PC/PS/PE phospholipid vesicles, was monitored every 8 s for 60 min. Recombinant human full-length TFPIα expressed in E. coli (kind gift by Tilman Hackeng, Maastricht, The Netherlands) was used at a final concentration of 5 nM to inhibit the amidolytic activity of FXa. We chose experimental conditions under which FXa was weakly inhibited by TFPIα alone but under which rhPS (final concentration, 20 nM) effectively enhanced FXa inhibition by TFPIα (FIG. 10A). In the absence of TFPIα, rhPS had not effect on the amidolytic activity FXa (data not shown).

The ability of rhPS to enhance the inhibitory activity of TFPIα was abolished when rhPS was pre-incubated for 15 min at room temperature with a blocking rabbit polyclonal anti-PS antibody (α-PS) (DAKO, final concentration 0.5 μM), but not with rabbit IgG (DAKO, final concentration 0.5 μM (FIG. 10B).

The ability of rhPS to enhance the inhibitory activity of TFPIα was evaluated when rhPS was pre-incubated for 15 min at room temperature with PS003 and PS003biv, or their respective monovalent (KB013) and bivalent (KB004biv) control nanobodies (final concentration 10 μM). A kinetic constant (k_{obs}) for the inhibition of FXa by TFPIα under each condition was calculated from the progress curves, as previously described (Ndonwi et al. 2010). The results were expressed as percentage of the TFPIα-cofactor activity of rhPS in the absence of nanobodies (TBS), and three experiments were done in simplicate, and Unpaired Student's t-test was used as a statistical test.

The results showed that PS003 and PS003biv did not potentiate, but rather slightly inhibited, the TFPIα-cofactor activity of rhPS, in this in vitro functional assay (FIG. 10C).

Binding of PS003biv and PS003biv to Immobilized Recombinant Murine PS (rmPS)

As for rhPS, recombinant murine PS (rmPS) was expressed in HEK293 cells in the presence of 10 μg/mL vitamin K1, and purified by a two-step anion-exchange chromatography, as previously described (Fernandez et al. 2009). rmPS (60 μL at 10 μg/mL in TBS containing 5 mM CaCl₂) was immobilized onto 96-well NUNC Maxisorp plates, for 16 h at 4° C. Wells were washed with 3×200 μL of washing buffer (TBS containing 5 mM CaCl₂) and 0.1% Tween-20) and wells were blocked for 1 h at room temperature with TBS containing 5 mM CaCl₂ and 5% BSA. Wells were washed with 3×200 μL of washing buffer and increasing concentrations of PS003biv and PS004biv (0-50 nM in TBS containing 5 mM CaCl₂ and 1% BSA, 50 μL/well) were incubated for 1 h at room temperature. Wells were washed with 3×200 μL of washing buffer, and a peroxidase-conjugated polyclonal anti-HA tag antibody (Abcam, 2 μg/mL in TBS containing 5 mM CaCl₂ and 1% BSA, 50 μL/well) was incubated for 1 h at room temperature to detect the bound nanobodies. Wells were washed with 3×200 μL of washing buffer and 50 μL of TMB was added. Reactions were stopped by the addition of 50 μL of 2M H₂SO₄.

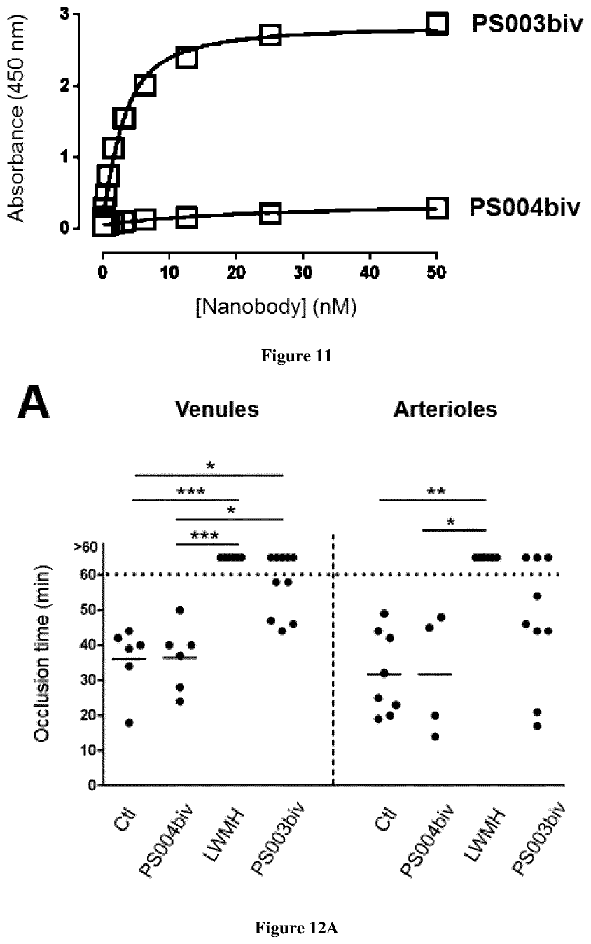

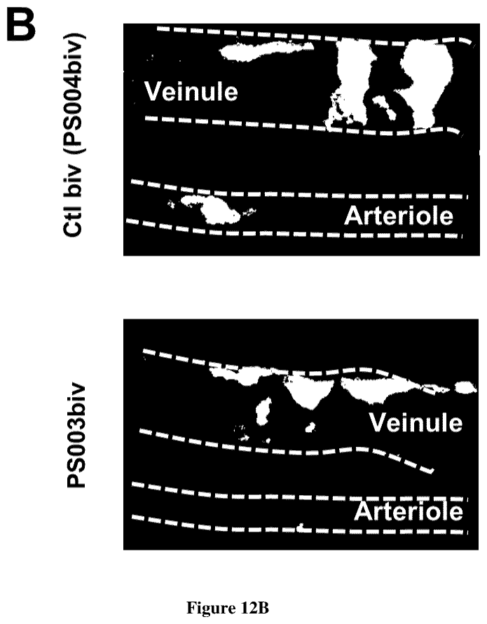

The results demonstrated that PS003biv strongly bound to rmPS (FIG. 11) and that PS003biv could be tested in thrombosis and bleeding models in mice. As PS004biv did not bind to rmPS in this assay (FIG. 11), it could potentially be used as a control nanobody for PS003biv in our in vivo models in mice.

As murine and human PS share 78% sequence homology between their SHBG-like regions, this result could also help localize the epitope for PS003biv. Indeed, candidate amino-acid residues are likely conserved within the SHBG-like region of human and murine PS, but not conserved within the the SHBG-like region of human Gas6.

In vivo antithrombotic effects of PS003biv in a mouse FeCl3-induced thrombosis model Ferric chloride (FeCl₃)-injury was induced in 4- to 5-week-old C57BL6/JRccHsd male mice, essentially as previously described (Aymé et al. 2017; Adam et al. 2010). To facilitate visualization of thrombus formation, platelets of anesthetized mice with pentobarbital were fluorescently labeled in vivo by intravenous injection of rhodamine 6G (3.3 mg/kg, i.e. 2.5 μL/g of rhodamine 6G at 1 mg/mL in 0.9% NaCl) into the retro-orbital plexus. PS003biv (10 mg/kg), PS004biv (10 mg/kg), or the same volumes of TBS buffer (Ctl), were diluted in 0.9% NaCl and were administered simultaneously. Alternatively, 200 UI/kg low-molecular-weight heparin (LMWH, Lovenox) was injected subcutaneously after the intravenous injection of rhodamine 6G alone, to verify that our thrombosis model was sensitive to pharmacological inhibition of coagulation. The labelled platelets were left to circulate for 10 min, and after a topical deposition of FeCl₃ solution (10% in water) on the mesenteric vessels, thrombus growth was monitored in real-time with an inverted epifluorescent microscope (×10) (Nikon Eclipse TE2000-U). One single venule and one single arteriole were analyzed for each mouse. Statistical analysis was assessed via Kruskal Wallis and Dunn's test. The results demonstrated that PS003biv exerted an antithrombotic effect in our FeCl₃-induced thrombosis model in mouse mesenteric vessels. Treatment of mice with PS003biv resulted in delayed occlusion, especially in the veinules (FIG. 12A). A similar trend was observed in the arterioles of mice treated with PS003biv, as compared to the control nanobody, but no statistical differences could be reached (FIG. 12A). The administration of PS003biv was also associated with thrombus stability and a higher rate of thrombus embolization (FIG. 12B). These apparent antithrombotic effects of PS003biv might, at least in part, reflect the enhancing effect of PS003biv on the APC-cofactor activity of rhPS.

It should be noted that the control bivalent anti-VWF nanobody (KB004biv) used in our APC- and TFPIα-cofactor activity assays could not be used in our FeCl₃-induced thrombosis model. Indeed, treatment of mice with this nanobody resulted in delayed occlusion times in the venule and arteriole of one mouse. Thus, we decided to use an in-house bivalent anti-PS nanobody (PS004biv) that is not able to bind to recombinant murine PS (FIG. 11).

Effects of PS003biv on Physiological Hemostasis in a Mouse Tail-Clip Bleeding Model

Anesthetized C57/BL6 mice were intravenously injected with PS003biv (10 mg/kg) or subcutaneously injected with low-molecular-weight heparin (LMWH) (Lovenox, 200 UI/kg), as described for the FeCl₃-induced thrombosis model. Tails were immerged at 37° C. for 10 min in 0.9% NaCl, and 3 mm from the tip of the tails were cut and immediately immerged at 37° C. into tubes containing 10 mL of 0.9% NaCl. Bleeding time was defined as the first cessation of bleeding. Blood was also collected during 20 minutes to quantify total blood loss volumes. Each bar represents the mean obtained from several mice evaluated. Ordinary one-way ANOVA was used as a statistical test of variance with Tukey's multiple comparison test.

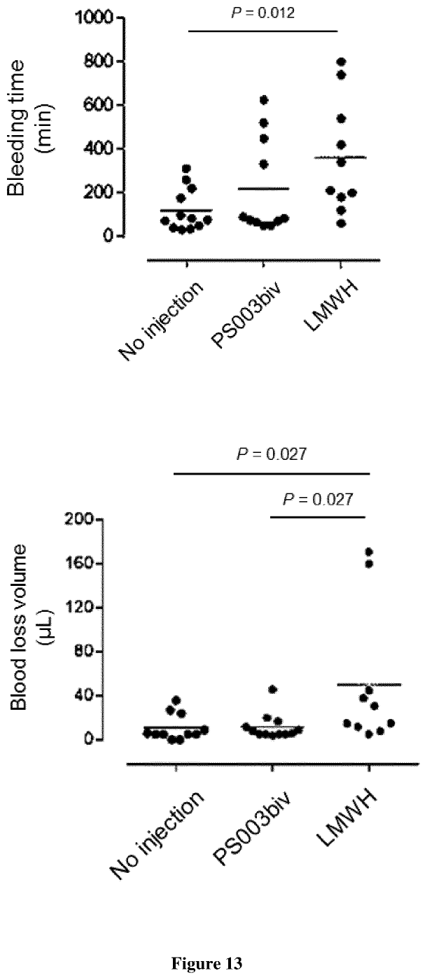

The results demonstrated that this murine bleeding model was sensitive to pharmacological inhibition of coagulation, as administration of 200 UI/kg low-molecular-weight heparin (LMWH) significantly prolonged the bleeding times and significantly increased the blood loss volumes (FIG. 13). This dose of LMWH also significantly prolonged the occlusion times in our murine FeCl₃-induced thrombosis model (FIG. 12A). In contrast to LMWH, PS003biv had no significant effect on both bleeding times and blood loss volumes (FIG. 13). These results supported our hypothesis that the enhancement of one of the anticoagulant activities of PS with the administration of PS003biv would not be associated with an impairment of physiological hemostasis. Consequently, our present study suggested the therapeutic potential of PS003biv as a potent and safe antithrombotic agent.

### Discussion

We propose that PS003/PS003biv nanobodies might have a therapeutic interest in sickle cell disease (SCD). SCD is a genetic disease resulting from a point mutation in the HBB gene, leading to polymerization of hemoglobin S (HbS) during deoxygenation, and to deformation of red blood cells into a sickle shape. Such sickling impairs red blood cell transit in microvessels and renders them prone to hemolysis. Red blood cell lysis releases noxious mediators that among others, activate vascular endothelial cells and drive the adhesion of leukocytes and platelets to the activated endothelium. These pathological events ultimately result in microvascular obstructions leading to the recurrent and painful vaso-occlusive crisis (VOC) that characterize SCD. These vaso-occlusive events can eventually result in end-organ damage and, in many cases, premature death.

The pathophysiology of VOC is complex and involves an interplay between sickled red blood cells, endothelial cells, platelets, and leukocytes. In addition, SCD patients are generally thought to be in a chronic hypercoagulable state (Whelihan et al. JTH 2016), as evidenced by the elevated levels of thrombin-antithrombin complexes (TAT), prothrombin fragment F1.2 and D-dimers in these patients (Ataga et al. Hematology Am Soc Hematol Educ Program 2007). This hypercoagulable state is associated with an increased risk of venous thromboembolism and stroke that has been well described in SCD patients (Sparkenbaugh and Pawlinksi. JTH 2017; Brunson et al. Br J Haematol 2017; Shet et al. Blood 2018). However, chronic coagulation activation in SCD might also locally trigger and/or enhance vascular inflammation, which is a crucial pathophysiological feature of SCD. Indeed, it has long been recognized that coagulation and inflammation can amplify one to another in various thrombo-inflammatory diseases, and this crosstalk between coagulation and inflammation is believed to be central to the pathophysiology of vaso-occlusion in SCD (Sparkenbaugh et al. Br J Haematol 2013).

Tissue factor (TF) expression has been shown to be increased in leukocytes in SCD patients and in mouse models of SCD, suggesting that TF likely contributes to hypercoagulability in SCD. Leukocyte TF is considered as the most likely source of TF contributing to coagulation activation in SCD (Sparkenbaugh and Pawlinski. JTH 2017), but TF is also inducibly expressed in vascular endothelial cells. Little is known about the role of contact system in hypercoagulability in SCD. FXII might be activated at sites of phosphatidylserine exposure on various cell types (e.g. sickled red blood cells and endothelial cells) and microvesicles derived from endothelial cells, platelets or monocytes. This can be inferred from a study showing that FXII is able bind to phosphatidylserine exposed by apoptotic cells, leading to its rapid cleavage and activation (Yang et al. Front Immunol 2017). Such an activation of FXII by phosphatidylserine could be an additional trigger of coagulation activation in SCD, independently of TF. Alternatively, FXII and contact system could be activated by mast cell-derived products, such as glycosaminoglycans and heparin, or by glycated hemoglobin released by hemolysis (Sparkenbaugh and Pawlinski. JTH 2017).

Exposure of phosphatidylserine at the surface of sickled red blood cells, endothelial cells, and microvesicles is an important driver of hypercoagulability in SCD. This exposure of phosphatidylserine considerably accelerates the rates of coagulation reactions and might also enhance decryption and activation of TF (Ansari et al. Thromb Haemost 2019). Interestingly, PS has a high affinity for phosphatidylserine-containing anionic phospholipid membranes, suggesting that PS might accumulate at these sites and might exert an important anticoagulant role to locally limit thrombin generation. However, widespread vascular and intravascular exposure of phosphatidylserine could also lead to capture of PS and to depletion from its plasma pool. This would be in line with the apparent acquired deficiency in PS observed in SCD patients in various studies (Whelihan et al. JTH 2016). Acute or chronic hypoxia might also participate in decreased plasma levels of PS observed in SCD patients, as liver expression of PS was found to be downregulated by hypoxia through HIF-1α (Pilli et al. Blood 2018). How such PS deficiency contributes to hypercoagulability and exacerbates prothrombotic tendency in SCD patients is not known. Interestingly, protein C deficiency was also found in SCD patients (Whelihan et al. JTH 2016), suggesting that the anticoagulant protein C pathway might be more broadly altered in SCD. Indeed, combined deficiency in PS and protein C in SCD is expected to have a great impact on the ability of activated protein C (APC) to exert its anticoagulant activity. This is supported by the APC resistance found in the plasma of SCD patients, even though elevated FVIII levels in these patients might also be a contributing factor (Wright et al. 1997; Whelihan et al. 2016).

Importantly, local coagulation activation and generation of thrombin might directly play a major role in the pathophysiology of VOC, independently of its ability to generate fibrin during thrombus formation. Indeed, thrombin not only cleaves fibrinogen to generate fibrin but is also a potent activator of endothelial cells, platelets, and leukocytes, notably through PAR1 activation. In endothelial cells, thrombin exerts PAR1-dependent pro-inflammatory, pro-apoptotic, and barrier-disruptive effects in endothelial cells (Flaumenhaft and De Ceunynck. Trends Pharmacol Sci 2017). Furthermore, thrombin-mediated activation of PAR1 on endothelial cells induces exocytosis of Weibel-Palade bodies containing Willebrand factor (VWF) and P-selectin (Cleator et al. Blood 2014), which contributes to or enhances the interaction between sickled red blood cells and endothelial cells. In addition, such exocytosis of Weibel-Palade bodies could release other soluble mediators of vascular thrombo-inflammation, such as angiopoietin-2. Furthermore, thrombin could directly or indirectly induce the exposure of phosphatidylserine on endothelial cells, thus fueling and perpetuating coagulation and thrombin generation at their surface.

Consequently, local and low-grade generation of thrombin initiated by TF and the contact system at the endothelial surface might be an early trigger of vascular inflammation and vaso-occlusive events, even in the absence of thrombus formation and widespread coagulation activation. Very recently, an anti-TF antibody, direct oral anticoagulants targeting FXa (rivaroxaban) and thrombin (dabigatran), and a PAR1 antagonist (vorapaxar) all markedly reduced hemoglobin-induced microvascular stasis in a mouse model of VOC (Sparkenbaugh et al. Blood 2020). Thus, pharmacological targeting of thrombin-mediated endothelial PAR1 activation appears as an attractive therapeutic strategy to prevent and/or reduce VOC in SCD. This study also suggests that a proper control of thrombin generation at the endothelial surface by natural anticoagulants such as PS, APC and tissue factor pathway inhibitor (TFPI), might be crucial to limit VOC.

PS exhibits high affinity for phosphatidylserine exposed on the surface of activated endothelial surface, and has a unique ability to function as a cofactor for both APC and TFPI-α. By stimulating the anticoagulant activities of both APC and TFPI-α, PS might have a central role in the limitation of thrombin generation at the surface of endothelial cells in SCD. Consequently, PS could be an important negative regulator of thrombin-induced vaso-occlusive events, even though this has yet to be corroborated in experimental studies.

We here propose that enhancing the APC-cofactor activity of PS with PS003/PS003biv nanobodies might constitute a novel therapeutic strategy for preventing or reducing vaso-occlusive events in SCD patients. Through their antithrombotic properties, PS003/PS003biv could concomitantly help reduce the risk of venous thromboembolic events and stroke in treated patients, especially since PS deficiency and APC resistance are found in SCD patients.

### REFERENCES

Throughout this application, various references describe the state of the art to which this invention pertains. The disclosures of these references are hereby incorporated by reference into the present disclosure.
Adam F, Kauskot A, Nurden P, Sulpice E, Hoylaerts M F, Davis R J, Rosa J P, Bryckaert M. Platelet JNK1 is involved in secretion and thrombus formation. Blood 2010; 115:4083-4092
Ansari S A, Pendurthi U R, Vijaya L, Rao M. Role of cell surface lipids and thiol-disulphide exchange pathways in regulating the encryption and decryption of tissue factor. Thromb Haemost 2019; 119:860-870
Ataga K I, Key N S. Hypercoagulability in sickle cell disease: new approaches to an old problem. Hematology Am Soc Hematol Educ Program 2007; 91-96
Aymé G, Adam F, Legendre P, Bazaa A, Proulle V, Denis C V, Christophe O D, Lenting P J. A novel single-domain antibody against Von Willebrand Factor A1 domain resolves leukocyte recruitment and vascular leakage during inflammation. Arterioscler Thromb Vasc Biol 2017; 37:1736-1740
Beatty J D, Beatty B G, Vlahos W G. Measurement of monoclonal antibody affinity by non-competitive enzyme immunoassay. J Immunol Methods 1987; 100(1-2):173-179
Brunson A, Lei A, Rosenberg A S, White R H, Keegan T, Wun T. Increased incidence of VTE in sickle cell disease patients: risk factors, recurrence and impact on mortality. Br J Haematol 2017; 178:319-326
Cantwell A M, Di Cera E. Rational design of a potent anticoagulant thrombin. J Biol Chem 2000; 275:39827-39830
Clauser S, Meilhac O, Biéche I, Raynal P, Bruneval P, Michel J B, Borgel D. Increased secretion of Gas6 by smooth muscle cells in human atherosclerotic carotid plaques. Thromb Haemost 2012; 107:140-149
Cleator J H, Zhu W Q, Vaughan D E, Hamm E E. Differential regulation of endothelial exocytosis of P-selectin and von Willebrand factor by protease-activated receptors and cAMP. Blood 2006; 107:2736-2744
Dargaud Y, Scoazec J Y, Wielders S J H, Trzeciak C, Hackeng T M, Négrier C, Hemker H C, Lindhout T, Castoldi E. Characterization of an autosomal dominant bleeding disorder caused by a thrombomodulin mutation. Blood 2015; 125:1497-1501
Fernandez J A, Kojima K, Petaja J, Hackeng T M, Griffin J H. Cardiolipin enhances protein C pathway anticoagulant activity. Blood Cells Mol Dis 2000; 26:115-123
Fernandez J A, Heeb M J, Xu X, Singh I, Zlokovic B V, Griffin J H. Species-specific anticoagulant and mitogenic activities of murine protein S. Haematologica 2009; 94:1721-1731
Flaumenhaft R, De Ceunynck K. Targeting PAR1: Now What? Trends Pharmacol Sci 2017; 38:701-716
Griffin J H, Kojima K, Banka C L, Curtiss L K, Fernandez J A. High-density lipoprotein enhancement of anticoagulant activities of plasma protein S and activated protein C. J Clin Invest 1999; 103:219-227
Gruber A, Cantwell A M, Di Cera E, Hanson S R. The thrombin mutant W215A/E217A shows safe and potent anticoagulant and antithrombotic effects in vivo. J Biol Chem 2002; 277:27581-27584
Gruber A, Marzec U M, Bush L, Di Cera E, Fernandez J A, Berny M A, Tuker E I, McCarty O J T, Griffin J H, Hanson S R. Relative antithrombotic and antihemostatic effects of protein C activator versus low-molecular-weight heparin in primates. Blood 2007; 109:3733-3740
Heeb M J, Fernandez J A, Yamashita A, McDowell O R, Guo Z, Mosnier L O, Deguchi H, Griffin J H. Activated protein C anticoagulant activity is enhanced by skeletal muscle myosin. Haematologica 2020; 105:e424-e427
McArthur J G, Svenstrup N, Chen C, Fricot A, Carvalho C, Nguyen J, Nguyen P, Parachikova A, Abdulla F, Vercellotti G M, Hermine O, Edwards D, Ribeil J A, Belcher J D, Maciel T T. A novel, highly potent and selective phosphodiesterase-9 inhibitor for the treatment of sickle cell disease. Haematologica 2020; 105:623-631
Ndonwi M, Tuley E A, Broze Jr G J. The Kunitz-3 domain of TFPI-α is required for protein S-dependent enhancement of factor Xa inhibition. Blood 2010; 116:1344-1351.
Saposnik B, Borgel D, Aiach M, Gandrille S. Functional properties of the Sex-Hormone-Binding Globulin (SHBG)-like domain of the anticoagulant protein S. Eur J Biochem 2003; 270:545-555
Shet A S, Wun T. How I diagnose and treat venous thromboembolism in sickle cell disease. Blood 2018; 132:1761-1769
Sparkenbaugh E, Pawlinski R. Interplay between coagulation and vascular inflammation in sickle cell disease. Br J Haematol 2013; 162:3-14
Sparkenbaugh E, Pawlinski R. Prothrombotic aspects of sickle cell disease. J Thromb Haemost 2017; 15:1307-1316
Sparkenbaugh E M, Chen C, Brzoska T, Nguyen J, Wang S, Vercellotti G M, Key N S, Sundd P, Belcher J D, Pawlinski R. Thrombin activation of PAR-1 contributes to microvascular stasis in mouse models of sickle cell disease. Blood 2020; 135:1783-1787
Studer R A, Opperdoes F R, Nicolaes G A F, Mulder A B, Mulder R. Understanding the functional difference between Growth arrest-specific protein 6 and protein S: an evolutionary approach. Open Biol 2014; 4:140121
Tucker E I, Verbout N G, Markway B D, Wallisch M, Lorentz C U, Hinds M T, Shatzel J J, Pelc L A, Wood D C, McCarty O J T, Di Cera E, Gruber A. The protein C activator AB002 rapidly interrupts thrombus development in baboons. Blood 2020; 135:689-699
Whelihan M F, Lim M Y, Mooberry M J, Piegore M G, Ilich A, Wogu A, Cai J, Monroe D M, Ataga K I, Mann K G, Key N S. Thrombin generation and cell-dependent hypercoagulability in sickle cell disease. J Thromb Haemost 2016; 14:1941-1952
Wright J G, Cooper P, Malia R G, Kulozik A E, Vetter B, Thomas P, Preston F E, Serjeant G R. Activated protein C resistance in homozygous sickle cell disease. Br J Haematol 1997; 96:854-856
Yang A, Chen F, He C, Zhou J, Lu Y, Dai J, Birge R B, Wu Y. The procoagulant activity of apoptotic cells is mediated by interaction with factor XII. Front Immunol 2017; 8:1188

1. An single-domain antibody (sdAb) directed against protein S (PS), wherein the single-domain antibody enhances APC-cofactor activity of PS.

2. The single-domain antibody according to claim 1, wherein said single-domain antibody comprises the CDR1 having the sequence set forth as SEQ ID NO: 1, the CDR2 having the sequence set forth as SEQ ID NO: 2 and the CDR3 having the sequence set forth as SEQ ID NO: 3.

3. The single-domain antibody according to claim 1 having at least 70% identity with the sequence set forth as SEQ ID NO 4.

4. The single-domain antibody according to claim 1 which comprises the sequence set forth as SEQ ID NO: 4.

5. A cross-competing single-domain antibody which cross-competes for binding PS with the single domain antibody according to claim 1.

6. A polypeptide comprising at least one single-domain antibody according to claim 1.

7. The polypeptide according to claim 6 which comprises at least two single-domain antibodies which enhance the APC-cofactor activity of PS.

8. The polypeptide according to claim 6, comprising two single-domain antibodies which enhance the APC-cofactor activity of PS.

9. The polypeptide according to claim 7 which comprises the sequence set forth as SEQ ID NO:5.

10. A nucleic acid molecule encoding i) the single-domain antibody of claim 1 and/or ii) a polypeptide comprising at least one single-domain antibody of claim 1.

11. A vector that comprises the nucleic acid of claim 10.

12. A host cell which has been transfected, infected or transformed by j the nucleic acid of claim 10 and/or ii) a vector comprising the nucleic acid of claim 10.

13. A method for preventing or treating a thrombotic disorders in a subject in need thereof, comprising administering to said subject an effective amount of the single-domain antibody of claim 1 and/or a polypeptide comprising at least one single-domain antibody according to claim 1.

14. A method for preventing or treating vaso-occlusive crisis in a subject in need thereof, comprising administering to said subject an effective amount of the single-domain antibody of claim 1 and/or a polypeptide comprising at least one single-domain antibody according to claim 1.

15. The method of claim 13, wherein the thrombotic disorders is sepsis, sickle-cell anemia, embolism, stroke or cardiovascular disease.

16. A pharmaceutical composition comprising the single-domain antibody of claim 1 and/or a polypeptide comprising at least one single-domain antibody according to claim 1.

17. The method of claim 15, wherein the embolism is a pulmonary or cerebral embolism.

18. The single-domain antibody of claim 1, wherein the single-domain antibody is an isolated single-domain antibody.


SEQUENCE LISTING (6 sequences)

SEQ ID NO: 1 - protein, 9 aa, Artificial Sequence
  Synthetic PS003 CDR1
SGRTFSSYA

SEQ ID NO: 2 - protein, 8 aa, Artificial Sequence
  Synthetic PS003 CDR2
ISYNGGRT

SEQ ID NO: 3 - protein, 15 aa, Artificial Sequence
  Synthetic PS003 CDR3
AANPRMWGSVDFRSW

SEQ ID NO: 4 - protein, 121 aa, Artificial Sequence
  Synthetic PS003
QVQLQESGGGLVQAGGSLRLSCAASGRTFSSYAMGWVRQAPGKEREFVAAISYNGGRTNYADSVKGRFTISRDNAKNTGYLQMNSLKPEDTAVYYCAANPRMWGSVDFRSWGQGTQVTVSS

SEQ ID NO: 5 - protein, 258 aa, Artificial Sequence
  Synthetic PS003biv
QVQLQESGGGLVQAGGSLRLSCAASGRTFSSYAMGWVRQAPGKEREFVAAISYNGGRTNYADSVKGRFTISRDNAKNTGYLQMNSLKPEDTAVYYCAANPRMWGSVDFRSWGQGTQVTVSSGGGSGGGSGGGSGGGSQVQLQESGGGLVQAGGSLRLSCAASGRTFSSYAMGWVRQAPGKEREFVAAISYNGGRTNYADSVKGRFTISRDNAKNTGYLQMNSLKPEDTAVYYCAANPRMWGSVDFRSWGQGTQVTVSS

SEQ ID NO: 6 - protein, 16 aa, Artificial Sequence
  Synthetic Linker
GGGSGGGSGGGSGGGS